(19) United States
(12) Patent Application Publication
(10) Pub. No.: US 2008/0311191 A1
(43) Pub. Date: Dec. 18, 2008

(54) Multi-Layer Tablets and Bioadhesive Dosage Forms

(76) Inventors: Avinash Nangia, Sharon, MA (US); Jules Jacob, Taunion, MA (US); Edith Mathiowitz, Brookline, MA (US); Thomas Ricketts, Halifax, MA (US); Mark R. Kreitz, Tacoma, WA (US)

Correspondence Address: ROPES & GRAY LLP, PATENT DOCKETING 39/41, ONE INTERNATIONAL PLACE, BOSTON, MA, 02110-2624, US

(21) Appl. No.: 11/661,541

(22) PCT Filed: Aug. 29, 2005
(86) PCT No.: PCT/US05/30651

§ 371 Date: Jan. 28, 2008

Related U.S. Application Data
(60) Provisional application No. 60/604,991, filed on Aug. 27, 2004.
(60) Provisional application No. 60/604,990, filed on Aug. 27, 2004.
(60) Provisional application No. 60/605,198, filed on Aug. 27, 2004.
(60) Provisional application No. 60/605,199, filed on Aug. 27, 2004.
(60) Provisional application No. 60/605,200, filed on Aug. 27, 2004.
(60) Provisional application No. 60/605,201, filed on Aug. 27, 2004.
(60) Provisional application No. 60/607,905, filed on Sep. 8, 2004.
(60) Provisional application No. 60/638,512, filed on Dec. 22, 2004.
(60) Provisional application No. 60/650,191, filed on Feb. 4, 2005.
(60) Provisional application No. 60/630,375, filed on Nov. 23, 2004.
(60) Provisional application No. 60/676,383, filed on Apr. 29, 2005.

(30) Foreign Application Priority Data
Dec. 9, 2004 (US) 11009327

(51) Int. Cl. A61K 9/52; A61K 9/32; A61K 9/48; A61K 9/24

(52) U.S. Cl. 424/457; 424/482; 424/463; 424/472

(57) ABSTRACT

Bioadhesives coatings increase the gastrointestinal retention time of orally-ingested medicaments. Certain bioadhesive coatings producing a fracture strength of at least 100 N/m², as measured on rat intestine, when applied to at least one surface of a pharmaceutical dosage form for oral delivery of a drug, result in a gastrointestinal retention time of at least 4 hours in a fed beagle dog model, during which the drug is released from the dosage form.

Multi-layer tablets, particularly those including hydrophobic excipients, are useful in administering hygroscopic and/or deliquescent drugs. In addition, varying the amount of drug in multi-layer tablets allows the release rate of the drug to be controlled.

### RELATED APPLICATIONS

This application claims the benefit of U.S. Provisional Application Nos. 60/604,990, filed Aug. 27, 2004, 60/604,991, filed Aug. 27, 2004, 60/605,198, filed Aug. 27, 2004, 60/605,199, filed Aug. 27, 2004, 60/605,200, filed Aug. 27, 2004, 60/605,201, filed Aug. 27, 2004, 60/607,905, filed Sep. 8, 2004, 60/635,812, filed Dec. 13, 2004, 60/650,191, filed Feb. 4, 2005, 60/650,375, filed Feb. 4, 2005 and 60/676,383, filed Apr. 29, 2005. This application is a continuation-in-part of U.S. application Ser. No. 11/009,327, filed Dec. 9, 2004 and a continuation-in-part of International Application No. PCT/US2005/007525, filed Mar. 3, 2005 in English and designating the U.S. The entire teachings of the above-referenced applications are incorporated herein by reference.

### BACKGROUND OF THE INVENTION

Controlled release systems for drug delivery are often designed to administer drugs in specific areas of the body. In the case of drug delivery to or via the gastrointestinal tract, it is critical that the drug not be delivered substantially beyond the desired site of action or absorption, respectively, before it has had a chance to exert a topical effect or to pass into the bloodstream. A drug delivery system that adheres to the lining of the appropriate viscus, will deliver its contents to the targeted tissue as a function of proximity and duration of contact.

An orally ingested product can adhere to either the epithelial surface or the mucus lining of the gastrointestinal tract. For the delivery of bioactive substances, it can be advantageous to have a polymeric drug delivery device adhere to the epithelium or to the mucous layer. Bioadhesion in the gastrointestinal tract proceeds in two stages: (1) viscoelastic deformation at the point of contact of the synthetic material into the mucus substrate, and (2) formation of bonds between the adhesive synthetic material and the mucus or the epithelial cells. In general, adhesion of polymers to tissues may be achieved by (i) physical or mechanical bonds, (ii) primary or covalent chemical bonds, and/or (iii) secondary chemical bonds (i.e., ionic). Physical or mechanical bonds can result from deposition and inclusion of the adhesive material in the crevices of the mucus or the folds of the mucosa. Secondary chemical bonds, contributing to bioadhesive properties, consist of dispersive interactions (i.e., Van der Waals interactions) and stronger specific interactions, which include hydrogen bonds. The hydrophilic functional groups primarily responsible for forming hydrogen bonds are the hydroxyl and the carboxylic acid groups.

Duchene et al., in Drug Dev. Ind. Pharm., 14:283-318 (1988), review the pharmaceutical and medical aspects of bioadhesive systems for drug delivery. Polycarbophils and acrylic acid polymers were noted as having the best adhesive properties. Others have explored the use of bioadhesive polymers, however, the extent of bioadhesion achieved in these studies has been limited. In addition, these studies do not demonstrate how to prepare larger bioadhesive drug delivery devices, such as tablets. WO 93/21906 discloses methods for fabricating bioadhesive microspheres and for measuring bioadhesive forces between microspheres and selected segments of the gastrointestinal tract in vitro. Lehr et al. screened microparticles formed of copolymers of acrylic acid using an in vitro system and determined that the copolymer “Polycarbophil” has increased adhesion.

Although bioadhesive-coated microparticles are known, larger oral formulations such as tablets with the ability to adequately adhere to the gastrointestinal tract mucosa are not known. The larger oral formulations differ from microparticles in that dosage forms such as tablets, capsules and drug-eluting devices cannot enter into an invagination in the mucosa, whereas microparticles are generally small enough to fit into an invagination. As a result, larger oral formulations contact a smaller surface area of the gastrointestinal tract (particularly as a function of the ratio of contact surface area to volume of the formulation), which is expected to weaken the interaction between the larger formulation and the gastrointestinal tract.

Separately or in addition to the need to control the location at which a drug is released, there is also a need to control the duration over which a drug is released from a pharmaceutical formulation. In particular, certain drugs, especially neuroactive drugs, have side effects and lower efficacy if blood serum concentrations vary considerably. Standard immediate release formulations typically cause such fluctuations in blood serum concentrations, because they dump large quantities of drug at one time into the patient's gastrointestinal tract.

Thus, there is a need for methods for controlling or increasing the absorption of pharmaceutical agents from drug delivery systems such as tablets through mucosal membranes. There also is a need for methods for delaying transit of pharmaceutical formulations through gastrointestinal passages.

### SUMMARY OF THE INVENTION

In one aspect, the present invention provides pharmaceutical dosage forms for oral delivery of a drug, comprising a drug to be delivered gastrointestinally, and a bioadhesive polymeric coating applied to at least a fraction of one surface of the dosage form. The coating provides the dosage form with a fracture strength of at least 100 N/m² as measured on rat intestine, and the dosage form has a gastrointestinal retention time of at least 4 hours in a fed beagle dog model during which the drug is released from the dosage form.

In a first embodiment, the present invention is a tablet for oral delivery of a drug, comprising a core including a drug to be delivered gastrointestinally, and a bioadhesive polymeric coating applied to at least one surface of the tablet. The coating provides the tablet with a fracture strength of at least 100 N/m² as measured on rat intestine, and the tablet has a gastrointestinal retention time of at least 4 hours in a fed beagle dog model during which the drug is released from the tablet. In certain embodiments, the bioadhesive polymer coating further includes metal compounds, low molecular weight oligomers or a combination thereof that enhance the mucosal adhesion of the synthetic polymer coating. In a preferred embodiment, the bioadhesive polymeric coating does not substantially swell upon hydration.

In one embodiment, the present invention is a tablet for oral delivery of a drug, comprising a core including a drug to be delivered gastrointestinally, and a bioadhesive polymeric coating applied to at least one surface of the tablet. The coating provides the tablet with a fracture strength of at least 100 N/m² as measured on rat intestine, and the tablet has a gastrointestinal retention time of at least 3 hours in a fasted beagle dog model (see Example 1) during which the drug is released from the tablet. In certain embodiments, the bioadhesive polymer coating further includes metal compounds, low molecular weight oligomers or a combination thereof that enhance the mucosal adhesion of the synthetic polymer coating. In a preferred embodiment, the bioadhesive polymeric coating does not substantially swell upon hydration.

In another embodiment, the invention is a drug-eluting device for oral delivery of a drug, which includes a reservoir having a drug-containing core contained therein, one or more orifices or exit ports through which drug from the core can elute from the device, and a bioadhesive polymeric coating, applied to at least one surface of the device. The coating provides the device with a fracture strength of at least 100 N/m² as measured on rat intestine, and the device has a gastrointestinal retention time of at least 3 hours in a fasted beagle dog model during which the drug is released from the device. In a preferred embodiment, the bioadhesive polymeric coating does not substantially swell upon hydration.

In yet another embodiment, the invention is a drug-eluting device for oral delivery of a drug, which includes a reservoir having a drug-containing core contained therein, one or more orifices or exit ports through which drug from the core can elute from the device, and a bioadhesive polymeric coating, applied to at least one surface of the device. The coating provides the device with a fracture strength of at least 100 N/m² as measured on rat intestine, and the device has a gastrointestinal retention time of at least 4 hours in a fed beagle dog model during which the drug is released from the device. In a preferred embodiment, the bioadhesive polymeric coating does not substantially swell upon hydration.

The present invention provides methods for improving the bioadhesive properties of drug delivery systems such as tablets, capsules and drug-eluting devices. The invention also provides methods for improving the adhesion of drug delivery systems to mucosal membranes including membranes of the gastrointestinal tract. The polymeric drug delivery systems of the invention have an improved ability to bind to mucosal membranes, and thus can be used to deliver a wide range of drugs or diagnostic agents in a wide variety of therapeutic applications, and/or improve uptake of the active agent across the intestinal mucosa. In certain embodiments, the drug delivery system comprises particles ranging in size from 0.1-10 μm.

Bioadhesive dosage forms of the invention generally have the advantages, inter alia, of allowing for decreasing dosage levels and/or dosing frequencies of drugs. Typically, the bioadhesive and/or mucoadhesive systems for the local and sustained delivery of therapeutic agents allow for more efficient targeting of drugs to the required sites on the luminal surface of the gastrointestinal tract. With the reduction of dosage level and/or dosing frequencies, several potential problems relating to antimicrobial agents may be reduced or avoided altogether, such as gastrointestinal irritation in some patients. Reduction of dosage level and/or dosing frequencies can also reduce or avoid disturbances of the normal enteric flora, which are caused by certain drugs, that may lead to drug-resistant bacterial enteritis or bacterial super-infection. The potential reduction in side effects and the overall ease of administration should greatly increase patient compliance, is expected to further improve the therapeutic outcome

In another embodiment, the present invention is an orally administrable, multi-layer, pharmaceutical tablet having an inner and one or more outer layers, each comprising a drug (e.g., a drug including a valproic moiety such as sodium valproate, divalproex sodium, valproic acid, etc.) admixed with one or more excipients. At least one of the excipients is hydrophobic, although such excipient is not required in each layer. Additional outer layers (i.e., layers other than the inner and outer layers specified above) are optionally free of the drug.

### BRIEF DESCRIPTION OF THE DRAWINGS

FIG. 1 is an illustration of a trilayer tablet with a bioadhesive coating.

FIGS. 2A-2D show that the trilayer tablets described in Example 1 were retained in the stomach of beagle dogs at A) 2.5 hours (fasted animal and tablet with Spheromer™ III, a poly(butadiene-co-maleic acid) functionalized with DOPA, outer layers), B) 3.5 hours (fasted animal and tablet with Spheromer™ III outer layers), C) 5.25 hours (fed animal and tablet with Spheromer™ I, poly(fumaric-co-sebacic anhydride 20:80), outer layers) and D) 6 hours (fed animal and tablet with Spheromer™ I outer layers).

FIG. 3 shows the pharmacokinetics of the 5-layer tablet in fed beagle dogs, as described in Example 2.

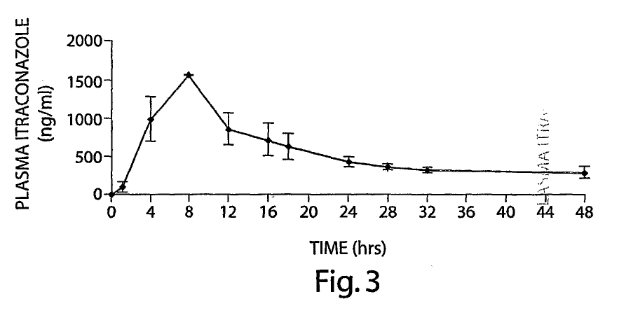

FIGS. 4A and 4B show the release profile of sodium valproate from the tablets prepared in Example 3.

FIG. 5 shows the weight gain experienced by sodium valproate trilayer tablet formulations of Example 3 (n=6) and a single-layer, matrix tablet formulation (n=6) consisting of only the core layer of the trilayer tablets when incubated at 45° C. and 60% relative humidity for up to 56 hours.

FIG. 6 shows the release profile of levodopa from the tablet prepared in Example 4.

FIG. 7 shows the effect of repeat dosing of the trilayer tablet of Example 5 (400 mg acyclovir, administered once per 12 hrs, 2 administrations) compared to Zovirax® (200 mg, administered once per 6 hrs, 4 administrations) in 6 dogs.

FIG. 8 shows the release profile of itraconazole from the tablet prepared in Example 6.

FIG. 9 shows the plasma levels of itraconazole following administration of tablets of Example 6 and Sporanox® to beagle dogs in the fed state, as measured using LC/MS/MS.

FIG. 10 is a bar graph showing the fracture strength of bonds (mN/cm²) formed with the bioadhesive materials, Spheromer™ II and Spheromer™ III, as compared to Carbopol 934P and Gantrez AN polymers and control (uncoated substrate).

FIG. 11 is a bar graph of the tensile work (nJ) required to rupture the bonds formed with the bioadhesive materials, Spheromer™ II and Spheromer™ III, as compared to Carbopol 934P and Gantrez AN polymers and control (uncoated substrate).

### DETAILED DESCRIPTION OF THE INVENTION

### Bioadhesive Pharmaceutical Dosage Forms

In one aspect, the present invention is directed to pharmaceutical dosage forms (e.g., tablets and drug-eluting devices) having increased gastrointestinal retention time. For purposes of this invention, gastrointestinal residence time is the time required for a pharmaceutical dosage form (e.g., tablet or drug-eluting device) to transit through the stomach to the pyloric sphincter. For example, a pharmaceutical dosage form (e.g., tablet or drug-eluting device) of the invention has a gastrointestinal residence time of at least 3 hours, at least 4 hours, at least 6 hours, at least 8 hours, or at least 12 hours. This time can be measured in either a fed or a fasted state, typically a fed state. The pharmaceutical dosage forms (e.g, tablets and drug-eluting devices) of the invention may also have an increased retention time in the small and/or large intestine, or in the area of the gastrointestinal tract that absorbs the drug contained in the pharmaceutical dosage form (e.g., tablet or drug-eluting device). For example, pharmaceutical dosage forms (e.g., tablets or drug-eluting devices) of the invention can be retained in the small intestine (or one or two portions thereof, selected from the duodenum, the jejunum and the ileum) for at least 6 hours, at least 8 hours or at least 12 hours, such as from 16 to 18 hours. For pharmaceutical dosage forms (e.g., tablets and drug-eluting devices) having an enteric coating or an equivalent, the increased gastrointestinal residence time may not be applicable, as the bioadhesive may not be exposed until the dosage form enters the small intestine or lower. These pharmaceutical dosage forms (e.g., tablets and drug-eluting devices), as a whole, include a bioadhesive polymeric coating that is applied to at least one surface of the dosage form.

“Bioadhesion” is defined as the ability of a material to adhere to a biological tissue for an extended period of time. Bioadhesion is one solution to the problem of inadequate residence time resulting from stomach emptying and intestinal peristalsis, and from displacement by ciliary movement. For sufficient bioadhesion to occur, an intimate contact must exist between the bioadhesive and the receptor tissue, the bioadhesive must penetrate into the crevice of the tissue surface and/or mucus, and mechanical, electrostatic, or chemical bonds must form. Bioadhesive properties of polymers are affected by both the nature of the polymer and by the nature of the surrounding media.

One example of a bioadhesive delivery system of the invention is for the local delivery of antimicrobial and acid lowering agents to eradicate Helicobacter pylori. Eradication of H. pylori not only cures both the gastric and duodenal ulcers but also has the potential to prevent a substantial proportion of gastric adenocarcinoma and lymphomas. In one embodiment, one or more therapeutic agents including acid suppressants (ranitidine bismuth citrate, lansoprazole), mucosal defense enhancing agent (bismuth salts) and/or mucolytic agents (megaldrate) are incorporated in the bioadhesive delivery system and then administered to patients with or at risk of H. pylori infection or ulcers. Preferably, the bioadhesive formulation includes a multi-layer core enveloped by a bioadhesive coating.

### Polymers

Suitable bioadhesive polymeric coatings are disclosed in U.S. Pat. Nos. 6,197,346, 6,217,908 and 6,365,187, the contents of which are incorporated herein by reference, and include soluble and insoluble, biodegradable and nonbiodegradable polymers. These can be hydrogels or thermoplastics, homopolymers, copolymers or blends, and/or natural or synthetic polymers. The preferred polymers are synthetic polymers, with controlled synthesis and degradation characteristics. Particularly preferred polymers are anhydride copolymers of fumaric acid and sebacic acid (P(FA:SA)), which have exceptionally good bioadhesive properties when administered to the gastrointestinal tract. Examples of P(FA:SA) copolymers include those having a 1:99 to 99:1 ratio of fumaric acid to sebacic acid, such as 5:95 to 75:25, for example, 10:90 to 60:40 or at least 15:85 to 25:75. Specific examples of such copolymers have a 20:80 or a 50:50 ratio of fumaric acid to sebacic acid.

Polymers used in bioadhesive pharmaceutical dosage forms (e.g., tablets and drug-eluting devices) of the invention produce a bioadhesive interaction (fracture strength) of at least 100 N/m² (10 mN/cm²) when applied to the mucosal surface of rat intestine. The fracture strength of the pharmaceutical dosage forms (e.g., tablets and drug-eluting devices) is advantageously at least 250 N/m², at least 500 N/m² or at least 1000 N/m². For example, the fracture strength of a polymer-containing pharmaceutical dosage form (e.g., tablet or drug-eluting device) can be from 100 to 500 N/m². The forces described herein refer to measurements made upon rat intestinal mucosa, unless otherwise stated. The same adhesive measurements made on other species of animal may differ from those obtained using rats. This difference is attributed to both compositional and geometrical variations in the mucous layers of different animal species as well as cellular variations in the mucosal epithelium. However, the data shows that the same general trends prevail across animals studied (i.e., P(FA:SA) produces stronger adhesions than polylactic acid (PLA) in rats, sheep, pigs, etc.). For example, the fracture strength of pharmaceutical dosage forms (e.g., tablets and drug-eluting devices) of the invention on rat intestine is generally at least 125 N/m², such as at least 150 N/m², at least 250 N/m², at least 500 N/m² or at least 1000 N/m².

The fracture strength of a pharmaceutical dosage form (e.g., tablet or drug-eluting device) can be measured according to the methods disclosed by Duchene et al. Briefly, the tablet is attached on one side to a tensile tester and is contacted with a testing surface (e.g., a mucosal membrane, such as rat or pig intestine) on the opposite surface. The tensile tester measures the force required to displace the pharmaceutical dosage form (e.g., tablet or drug-eluting device) from the testing surface. Common tensile testers include a Texture Analyzer and the Instron tensile tester.

In the preferred method for mucoadhesive testing, tablets are pressed using flat-faced tooling, 0.3750″ (9.525 mm) in diameter. Tablet weight will depend on composition; in most cases, the tablets have a final weight of 200 mg. These tablets are then glued to a plastic 10 mm diameter probe using a common, fast-drying cyanoacrylate adhesive. Once the tablets are firmly adhered to the probe, the probe is attached to the Texture Analyzer. The Texture Analyzer is fitted with a 1 kg load cell for maximum sensitivity. The following settings are used:

| Pre-Test Speed | 0.4 | mm/sec |
|---|---|---|
| Test Speed | 0.1 | mm/sec |
| Post-Test Speed | 0.1 | mm/sec |
| Applied Force | 20.0 | g |
| Return Distance | 0 | mm |
| Contact Time | 420 | s |
| Trigger Type | Auto | |
| Trigger Force | 0.5 | g |
| Stop Plot At | Final Position | |
| Tare Mode | Auto | |
| Delay Acquisition | Off | |
| Advanced Options | On | |
| Proportional Gain | 0 | |
| Integral Gain | 0 | |
| Differential Gain | 0 | |
| Max. Tracking Speed | 0 | mm/sec |

The Test and Post-Test Speeds are advantageously as low as the instrument permits, in order to allow capture of a maximum number of data points. The Pre-Test speed is used only until the probe encounters the Trigger Force; i.e., prior to contacting the tissue.

The Proportional, Integral, and Differential Gain are set to 0. These settings, when optimized, maintain the system at the Applied Force for the duration of the Contact Time. With soft tissue as a substrate, however, the probe and tablet are constantly driven into the deformable surface. This results in visible damage to the tissue. Thus, the probe and tablet are allowed to relax gradually from the Applied Force by setting these parameters to 0. The tracking speed, which is a measure of how rapidly the feedback is adjusted, is also set to 0.

The tissue on which the tablets are tested is secured in the Mucoadhesive Rig; the rig is then completely immersed in a 600 mL Pyrex beaker containing 375 mL of PBS. The tissue is maintained at approximately 37° C. for the duration of the test; no stirring is used as the machine can detect the oscillations from the stir bar.

Smart et al., J. Pharm. Pharmacol., 36:295-299 (1984), report another method to test adhesion to mucosa using a polymer coated glass plate contacting a dish of mucosa. A variety of polymeric materials were tested, including sodium alginate, sodium carboxymethyl-cellulose, gelatin, pectin and polyvinylpyrrolidone.

Gurney et al., Biomaterials, 5:336-340 (1984) report that adhesion may be affected by physical or mechanical bonds; secondary chemical bonds; and/or primary, ionic or covalent bonds. Park et al., “Alternative Approaches to Oral Controlled Drug Delivery: Bioadhesives and In-Situ Systems,” in J. M. Anderson and S. W. Kim, Eds., “Recent Advances in Drug Delivery,” Plenum Press, New York, 1984, pp. 163-183, report a study of the use of fluorescent probes in cells to determine adhesiveness of polymers to mucin/epithelial surface, which indicates that anionic polymers with high charge density appear to be preferred as adhesive polymers. Mikos et al., in J. Colloid Interface Sci., 143:366-373 (1991) and Lehr et al., J. Controlled Rel. Soc., 13:51-62 (1990) report a study of the bioadhesive properties of polyanhydrides and polyacrylic acid, respectively, in drug delivery.

In the past, two classes of polymers have shown useful bioadhesive properties, hydrophilic polymers and hydrogels. In the large class of hydrophilic polymers, those containing carboxylic groups (e.g., poly[acrylic acid]) exhibit the best bioadhesive properties. It is thus expected that polymers with the highest concentrations of carboxylic groups are preferred materials for bioadhesion on soft tissues. In other studies, the most promising polymers were sodium alginate, carboxymethylcellulose, hydroxymethylcellulose and methylcellulose. Some of these materials are water-soluble, while others are hydrogels.

Rapidly bioerodible polymers such as poly[lactide-co-glycolide], polyanhydrides, and polyorthoesters, whose carboxylic groups are exposed on the external surface as their smooth surface erodes, are particularly suitable for bioadhesive drug delivery systems. In addition, polymers containing labile bonds, such as polyanhydrides and polyesters, are well known for their hydrolytic reactivity. Their hydrolytic degradation rates can generally be altered by simple changes in the polymer backbone.

Representative natural polymers suitable for the present invention include proteins (e.g., hydrophilic proteins), such as zein, modified zein, casein, gelatin, gluten, serum albumin, or collagen, and polysaccharides such as cellulose, dextrans, polyhyaluronic acid, polymers of acrylic and methacrylic esters and alginic acid. These are generally less suitable for use in bioadhesive coatings due to higher levels of variability in the characteristics of the final products, as well as in degradation following administration. Synthetically modified natural polymers include alkyl celluloses, hydroxyalkyl celluloses, cellulose ethers, cellulose esters, and nitrocelluloses.

Representative synthetic polymers for use in bioadhesive coatings include polyphosphazines, poly(vinyl alcohols), polyamides, polycarbonates, polyalkylenes, polyacrylamides, polyalkylene glycols, polyalkylene oxides, polyalkylene terephthalates, polyvinyl ethers, polyvinyl esters, polyvinyl halides, polyvinylpyrrolidone, polyglycolides, polysiloxanes, polyurethanes and copolymers thereof. Other polymers suitable for use in the invention include, but are not limited to, methyl cellulose, ethyl cellulose, hydroxypropyl cellulose, hydroxypropyl methyl cellulose, hydroxybutyl methyl cellulose, cellulose acetate, cellulose propionate, cellulose acetate butyrate, cellulose acetate phthalate, carboxymethyl cellulose, cellulose triacetate, cellulose sulfate sodium salt, poly(methyl methacrylate), poly(ethyl methacrylate), poly(butyl methacrylate), poly(isobutyl methacrylate), poly(hexyl methacrylate), poly(isodecyl methacrylate), poly(lauryl methacrylate), poly(phenyl methacrylate), poly(methyl acrylate), poly(isopropyl acrylate), poly(isobutyl acrylate), poly(octadecyl acrylate) polyethylene, polypropylene, poly(ethylene glycol), poly(ethylene oxide), poly (ethylene terephthalate), poly(vinyl acetate), polyvinyl chloride, polystyrene, polyvinyl pyrrolidone, and polyvinylphenol. Representative bioerodible polymers for use in bioadhesive coatings include polylactides, polyglycolides and copolymers thereof, poly(ethylene terephthalate), poly(butyric acid), poly(valeric acid), poly(lactide-co-caprolactone), poly[lactide-co-glycolide], polyanhydrides (e.g., poly(adipic anhydride)), polyorthoesters, blends and copolymers thereof.

Polyanhydrides are particularly suitable for use in bioadhesive delivery systems because, as hydrolysis proceeds, causing surface erosion, more and more carboxylic groups are exposed to the external surface. However, polylactides erode more slowly by bulk erosion, which is advantageous in applications where it is desirable to retain the bioadhesive coating for longer durations. In designing bioadhesive polymeric systems based on polylactides, polymers that have high concentrations of carboxylic acid are preferred. The high concentrations of carboxylic acids can be attained by using low molecular weight polymers (MW of 2000 or less), because low molecular weight polymers contain a high concentration of carboxylic acids at the end groups.

The polymers listed above can be obtained from sources such as Sigma Chemical Co., St. Louis, Mo., Polysciences, Warrenton, Pa., Aldrich, Milwaukee, Wis., Fluka, Ronkonkoma, N.Y., and BioRad, Richmond, Calif., or can alternatively be synthesized from monomers obtained from these suppliers using standard techniques.

When the bioadhesive polymeric coating is a synthetic polymer coating, the synthetic polymer is typically selected from polyamides, polycarbonates, polyalkylenes, polyalkylene glycols, polyalkylene oxides, polyalkylene terephthalates, polyvinyl alcohols, polyvinyl ethers, polyvinyl esters, polyvinyl halides, polyvinylpyrrolidone, polyglycolides, polysiloxanes, polyurethanes, polystyrene, polymers of acrylic and methacrylic esters, polylactides, poly(butyric acid), poly(valeric acid), poly(lactide-co-glycolide), polyanhydrides, polyorthoesters, poly(fumaric acid), poly(maleic acid), and blends and copolymers of thereof. In an exemplary embodiment, the synthetic polymer is poly(fumaric-co-sebacic) anhydride.

Another group of polymers suitable for use as bioadhesive polymeric coatings are polymers having a hydrophobic backbone with at least one hydrophobic group pendant from the backbone. Suitable hydrophobic groups are groups that are generally non-polar. Examples of such hydrophobic groups include alkyl, alkenyl and alkynyl groups. Preferably, the hydrophobic groups are selected to not interfere and instead to enhance the bioadhesiveness of the polymers.

A further group of polymers suitable for use as bioadhesive polymeric coatings are polymers having a hydrophobic backbone with at least one hydrophilic group pendant from the backbone. Suitable hydrophilic groups include groups that are capable of hydrogen bonding or electrostatically bonding to another functional group. Example of such hydrophilic groups include negatively charged groups such as carboxylic acids, sulfonic acids and phosphonic acids, positively charged groups such as (protonated) amines and neutral, polar groups such as amides and imines. Preferably, the hydrophilic groups are selected to not interfere and instead to enhance the bioadhesiveness of the polymers. The hydrophilic groups can be either directly attached to a hydrophobic polymer backbone or attached through a spacer group. Typically, a spacer group is an alkylene group, particularly a C₁-C₈ alkyl group such as a C₂-C₆ alkyl group. Preferred compounds containing one or more hydrophilic groups include amino acids (e.g., phenyalanine, tyrosine and derivatives thereof) and amine-containing carbohydrates (sugars) such as glucosamine.

Polymers can be modified by increasing the number of carboxylic groups accessible during biodegradation, or on the polymer surface. The polymers can also be modified by binding amino groups to the polymer. The polymers can be modified using any of a number of different coupling chemistries available in the art to covalently attach ligand molecules with bioadhesive properties to the surface-exposed molecules of the polymeric microspheres.

Lectins can be covalently attached to polymers to render them target specific to the mucin and mucosal cell layer. Useful lectin ligands include lectins isolated from: Abrus precatroius, Agaricus bisporus, Anguilla anguilla, Arachis hypogaea, Pandeiraea simplicifolia, Bauhinia purpurea, Caragan arobrescens, Cicer arietinum, Codium fragile, Datura stramonium, Dolichos biflorus, Erythrina corallodendron, Erythrina cristagalli, Euonymus europaeus, Glycine max, Helix aspersa, Helix pomatia, Lathyrus odoratus, Lens culinaris, Limulus polyphemus, Lysopersicon esculentum, Maclura pomifera, Momordica charantia, Mycoplasma gallisepticum, Naja mocambique, as well as the lectins Concanavalin A, Succinyl-Concanavalin A, Triticum vulgaris, Ulex europaeus I, II and III, Sambucus nigra, Maackia amurensis, Limax fluvus, Homarus americanus, Cancer antennarius, and Lotus tetragonolobus.

The attachment of any positively charged ligand, such as polyethyleneimine or polylysine, to a polymer may improve bioadhesion due to the electrostatic attraction of the cationic groups coating the beads to the net negative charge of the mucus. The mucopolysaccharides and mucoproteins of the mucin layer, especially the sialic acid residues, are responsible for the negative charge coating. Any ligand with a high binding affinity for mucin could also be covalently linked to most polymers with the appropriate chemistry, such as with carbodiimidazole (CDI), and be expected to influence the binding to the gut. For example, polyclonal antibodies raised against components of mucin or else intact mucin, when covalently coupled to a polymer, would provide for increased bioadhesion. Similarly, antibodies directed against specific cell surface receptors exposed on the lumenal surface of the intestinal tract would increase the residence time when coupled to polymers using the appropriate chemistry. The ligand affinity need not be based only on electrostatic charge, but other useful physical parameters such as solubility in mucin or specific affinity to carbohydrate groups.

The covalent attachment of any of the natural components of mucin in either pure or partially purified form to the polymers generally increases the solubility of the polymer in the mucin layer. The list of useful ligands include but are not limited to the following: sialic acid, neuraminic acid, n-acetyl-neuraminic acid, n-glycolylneuraminic acid, 4-acetyl-n-acetylneuraminic acid, diacetyl-n-acetylneuraminic acid, glucuronic acid, iduronic acid, galactose, glucose, mannose, fucose, any of the partially purified fractions prepared by chemical treatment of naturally occurring mucin, e.g., mucoproteins, mucopolysaccharides and mucopolysaccharide-protein complexes, and antibodies immunoreactive against proteins or sugar structure on the mucosal surface.

The attachment of polyamino acids containing extra pendant carboxylic acid side groups, such as polyaspartic acid and polyglutamic acid, may also increase bioadhesiveness. The polyamino chains would increase bioadhesion by means of chain entanglement in mucin strands as well as by increased carboxylic charge.

### Polymer-Metal Complexes

As disclosed in U.S. Pat. Nos. 5,985,312, 6,123,965 and 6,368,586, the contents of which are incorporated herein by reference, polymers, such as those named above, having a metal compound incorporated therein have a further improved ability to adhere to tissue surfaces, such as mucosal membranes, and are suitable for use in the invention. The metal compound incorporated into the polymer can be, for example, a water-insoluble metal oxide. The incorporation of metal compounds into a wide range of different polymers, even those that are not normally bioadhesive, often improves their ability to adhere to tissue surfaces such as mucosal membranes.

Metal compounds that can be incorporated into polymers to improve their bioadhesive properties preferably are water-insoluble metal compounds, such as water-insoluble metal oxides and metal hydroxides, which are capable of becoming incorporated into and associated with a polymer to improve the bioadhesiveness of the polymer. As defined herein, a water-insoluble metal compound is defined as a metal compound with little or no solubility in water, for example, less than about 0.0 to 0.9 mg/ml.

The water-insoluble metal compounds can be derived from a wide variety of metals, including, but not limited to, calcium, iron, copper, zinc, cadmium, zirconium and titanium. The water-insoluble metal compound preferably is a metal oxide or hydroxide. Water-insoluble metal compounds of multivalent metals are preferred. Representative metal oxides suitable for use in the compositions described herein include cobalt (II) oxide (CoO), cobalt (III) oxide (CO₂O₃), selenium oxide (SeO₂), chromium (IV) oxide (CrO₂), manganese oxide (MnO₂), titanium oxide (TiO₂), lanthanum oxide (La₂O₃), zirconium oxide (ZrO₂), silicon oxide (SiO₂), scandium oxide (Sc₂O₃), beryllium oxide (BeO), tantalum oxide (Ta₂O₅), cerium oxide (CeO₂), neodymium oxide (Nd₂O₃), vanadium oxide (V₂O₅), molybdenum oxide (Mo₂O₃), tungsten oxide (WO), tungsten trioxide (WO₃), samarium oxide (Sm₂O₃), europium oxide (Eu₂O₃), gadolinium oxide (Gd₂O₃), terbium oxide (Tb₄O₇), dysprosium oxide (Dy₂O₃), holmium oxide (Ho₂O₃), erbium oxide (Er₂O₃), thulium oxide (Tm₂O₃), ytterbium oxide (Yb₂O₃), lutetium oxide (Lu₂O₃), aluminum oxide (Al₂O₃), indium oxide (InO₃), germanium oxide (GeO₂), antimony oxide (Sb₂O₃), tellurium oxide (TeO₂), nickel oxide (NiO), and zinc oxide (ZnO). Other oxides include barium oxide (BaO), calcium oxide (CaO), nickel (III) oxide (Ni₂O₃), magnesium oxide (MgO), iron (II) oxide (FeO), iron (III) oxide (Fe₂O₃), copper (II) oxide (CuO), cadmium oxide (CdO), and zirconium oxide (ZrO₂).

Preferred properties defining the metal compound include: (a) substantial insolubility in aqueous environments, such as acidic or basic aqueous environments (such as those present in the gastric lumen); and (b) ionizable surface charge at the pH of the aqueous environment.

The water-insoluble metal compounds can be incorporated into the polymer by one of the following mechanisms: (a) physical mixtures which result in entrapment of the metal compound; (b) ionic interaction between metal compound and polymer; (c) surface modification of the polymers which would result in exposed metal compound on the surface; and (d) coating techniques such as fluidized bed, pan coating, or any similar methods known to those skilled in the art, which produce a metal compound enriched layer on the surface. In one embodiment, nanoparticles or microparticles of the water-insoluble metal compound are incorporated into the polymer.

In one embodiment, the metal compound is provided as a fine particulate dispersion of a water-insoluble metal oxide e.g., incorporated throughout the polymer or disposed on the surface of the polymer which is to be adhered to a tissue surface. The metal compound also can be incorporated in an inner layer of the polymer and exposed only after degradation or else dissolution of a “protective” outer layer. For example, a tablet core containing a polymer and metal may be covered with an enteric coating designed to dissolve when exposed to gastric fluid. The metal compound-enriched core then is exposed and becomes available for binding to GI mucosa.

Fine metal oxide particles can be produced for example by micronizing a metal oxide by mortar and pestle treatment to produce particles ranging in size, for example, from 10.0 to 300 nm. The metal oxide particles can be incorporated into the polymer, for example, by dissolving or dispersing the particles into a solution or dispersion of the polymer.

Advantageously, metal compounds which are incorporated into polymers to improve their bioadhesive properties can be metal compounds which are already approved by the FDA as either food or pharmaceutical additives, such as zinc oxide.

Suitable polymers that can be used and into which the metal compounds can be incorporated include soluble and water-insoluble, and biodegradable and nonbiodegradable polymers, including hydrogels, thermoplastics, and homopolymers, copolymers and blends of natural and synthetic polymers, provided that they have the requisite fracture strength when mixed with a metal compound. In additional to those listed above, representative polymers which can be used in conjunction with a metal compound include hydrophilic polymers, such as those containing carboxylic groups, including polyacrylic acid. Bioerodible polymers including polyanhydrides, poly(hydroxy acids) and polyesters, as well as blends and copolymers thereof also can be used. Representative bioerodible poly(hydroxy acids) and copolymers thereof which can be used include poly(lactic acid), poly(glycolic acid), poly(hydroxy-butyric acid), poly(hydroxyvaleric acid), poly(caprolactone), poly(lactide-co-caprolactone), and poly(lactide-co-glycolide). Polymers containing labile bonds, such as polyanhydrides and polyorthoesters, can be used optionally in a modified form with reduced hydrolytic reactivity. Positively charged hydrogels, such as chitosan, and thermoplastic polymers, such as polystyrene also can be used.

Representative natural polymers which also can be used include proteins, such as zein, modified zein, casein, gelatin, gluten, serum albumin, or collagen, and polysaccharides such as dextrans, polyhyaluronic acid and alginic acid. Representative synthetic polymers include polyphosphazenes, polyamides, polycarbonates, polyacrylamides, polysiloxanes, polyurethanes and copolymers thereof. Celluloses also can be used. As defined herein the term “celluloses” includes naturally occurring and synthetic celluloses, such as alkyl celluloses, cellulose ethers, cellulose esters, hydroxyalkyl celluloses and nitrocelluloses. Exemplary celluloses include ethyl cellulose, methyl cellulose, carboxymethyl cellulose, hydroxymethyl cellulose, hydroxypropyl cellulose, hydroxypropyl methyl cellulose, hydroxybutyl methyl cellulose, cellulose acetate, cellulose propionate, cellulose acetate butyrate, cellulose acetate phthalate, cellulose triacetate and cellulose sulfate sodium salt.

Polymers of acrylic and methacrylic acids or esters and copolymers thereof can be used. Representative polymers which can be used include poly(methyl methacrylate), poly(ethyl methacrylate), poly(butyl methacrylate), poly(isobutyl methacrylate), poly(hexyl methacrylate), poly(isodecyl methacrylate), poly(lauryl methacrylate), poly(phenyl methacrylate), poly(methyl acrylate), poly(isopropyl acrylate), poly(isobutyl acrylate), and poly(octadecyl acrylate).

Other polymers which can be used include polyalkylenes such as polyethylene and polypropylene; polyarylalkylenes such as polystyrene; poly(alkylene glycols), such as poly(ethylene glycol); poly(alkylene oxides), such as poly(ethylene oxide); and poly(alkylene terephthalates), such as poly(ethylene terephthalate). Additionally, polyvinyl polymers can be used, which as defined herein includes polyvinyl alcohols, polyvinyl ethers, polyvinyl esters and polyvinyl halides. Exemplary polyvinyl polymers include poly(vinyl acetate), polyvinyl phenol and polyvinylpyrrolidone.

Water soluble polymers can also be used. Representative examples of suitable water soluble polymers include polyvinyl alcohol, polyvinylpyrrolidone, methyl cellulose, hydroxypropyl cellulose, hydroxypropylmethyl cellulose and polyethylene glycol, copolymers of acrylic and methacrylic acid esters, and mixtures thereof. Water insoluble polymers also can be used. Representative examples of suitable water insoluble polymers include ethylcellulose, cellulose acetate, cellulose propionate (lower, medium or higher molecular weight), cellulose acetate propionate, cellulose acetate butyrate, cellulose acetate phthalate, cellulose triacetate, poly(methyl methacrylate), poly(ethyl methacrylate), poly(butyl methacrylate), poly(isobutyl methacrylate), poly(hexyl methacrylate), poly(isodecyl methacrylate), poly(lauryl methacrylate), poly(phenyl methacrylate), poly(methyl acrylate), poly(isopropyl acrylate), poly(isobutyl acrylate), poly(octadecyl acrylate), poly(ethylene), poly(ethylene) low density, poly(ethylene) high density, poly(propylene), poly(ethylene oxide), poly(ethylene terephthalate), poly(vinyl isobutyl ether), poly(vinyl acetate), poly(vinyl chloride), polyurethanes, and mixtures thereof. In one embodiment, a water insoluble polymer and a water soluble polymer are used together, such as in a mixture. Such mixtures are useful in controlled drug release formulations, wherein the release rate can be controlled by varying the ratio of water soluble polymer to water insoluble polymer.

Polymers varying in viscosity as a function of temperature or shear or other physical forces also may be used. Poly(oxyalkylene) polymers and copolymers such as poly(ethylene oxide)-poly(propylene oxide) (PEO-PPO) or poly(ethylene oxide)-poly(butylene oxide) (PEO-PBO) copolymers, and copolymers and blends of these polymers with polymers such as poly(alpha-hydroxy acids), including but not limited to lactic, glycolic and hydroxybutyric acids, polycaprolactones, and polyvalerolactones, can be synthesized or commercially obtained. For example, polyoxyalkylene copolymers are described in U.S. Pat. Nos. 3,829,506, 3,535,307, 3,036,118, 2,979,578, 2,677,700 and 2,675,619. Polyoxyalkylene copolymers are sold, for example, by BASF under the tradename PLURONICS™. These materials are applied as viscous solutions at room temperature or lower which solidify at the higher body temperature. Other materials with this behavior are known in the art, and can be utilized as described herein. These include KLUCEL™ (hydroxypropyl cellulose), and purified konjac glucomannan gum.

Other suitable polymers are polymeric lacquer substances based on acrylates and/or methacrylates, commonly called EUDRAGIT™ polymers (sold by Rohm America, Inc.). Specific EUDRAGIT™ polymers can be selected having various permeability and water solubility, which properties can be pH dependent or pH independent. For example, EUDRAGIT™ RL and EUDRAGIT™ RS are acrylic resins comprising copolymers of acrylic and methacrylic acid esters with a low content of quaternary ammonium groups, which are present as salts and give rise to the permeability of the lacquer films, whereas EUDRAGIT™ RL is freely permeable and EUDRAGIT™ RS is slightly permeable, independent of pH. In contrast, the permeability of EUDRAGIT™ L is pH-dependent. EUDRAGIT™ L is an anionic polymer synthesized from methacrylic acid and methacrylic acid methyl ester. It is insoluble in acids and pure water, but becomes increasingly soluble in a neutral to weakly alkaline solution by forming salts with alkalis. Above pH 5.0, the polymer becomes increasingly permeable.

Polymer solutions that are liquid at an elevated temperature but solid or gelled at body temperature can also be utilized. A variety of thermoreversible polymers are known, including natural gel-forming materials such as agarose, agar, furcellaran, beta-carrageenan, beta-1,3-glucans such as curdlan, gelatin, or polyoxyalkylene-containing compounds, as described above. Specific examples include thermosetting biodegradable polymers for in vivo use described in U.S. Pat. No. 4,938,763, the contents of which are incorporated herein by reference.

Polymer Blends with Monomers and/or Oligomers

Polymers with enhanced bioadhesive properties are provided by incorporating anhydride monomers or oligomers into one of the polymers listed above by dissolving, dispersing, or blending, as taught by U.S. Pat. Nos. 5,955,096 and 6,156,348, the contents of which are incorporated herein by reference. The polymers may be used to form drug delivery systems which have improved ability to adhere to tissue surfaces, such as mucosal membranes. The anhydride oligomers are generally formed from organic diacid monomers, preferably the diacids normally found in the Krebs glycolysis cycle. Anhydride oligomers that enhance the bioadhesive properties of a polymer have a molecular weight of about 5000 or less, typically between about 100 and 5000 daltons, or include 20 or fewer diacid units linked by anhydride linkages and terminating in an anhydride linkage with a carboxylic acid monomer.

The oligomers can be blended or incorporated into a wide range of hydrophilic and hydrophobic polymers including proteins, polysaccharides and synthetic biocompatible polymers, including those described above. In one embodiment, anhydride oligomers may be combined with metal oxide particles, such as those described above, to improve bioadhesion even more than with the organic additives alone. Organic dyes, because of their electronic charge and hydrophobicity or hydrophilicity, can either increase or decrease the bioadhesive properties of polymers when incorporated into the polymers.

As used herein, the term “anhydride oligomer” refers to a diacid or polydiacid linked by anhydride bonds, and having carboxy end groups linked to a monoacid such as acetic acid by anhydride bonds. The anhydride oligomers have a molecular weight less than about 5000, typically between about 100 and 5000 daltons, or are defined as including between one to about 20 diacid units linked by anhydride bonds. In one embodiment, the diacids are those normally found in the Krebs glycolysis cycle.

The oligomers can, for example, be formed in a reflux reaction of the diacid with excess acetic anhydride. The excess acetic anhydride is evaporated under vacuum, and the resulting oligomer, which is a mixture of species which include from about one to twenty diacid units linked by anhydride bonds, is purified by recrystallizing, for example, from toluene or other organic solvents. The oligomer is collected by filtration, and washed, for example, in ethers. The reaction produces anhydride oligomers of mono and poly acids with terminal carboxylic acid groups linked to each other by anhydride linkages.

An anhydride oligomer is hydrolytically labile. As analyzed by gel permeation chromatography, the molecular weight may be, for example, on the order of 200-400 for fumaric acid oligomer (FAPP) and 2000-4000 for sebacic acid oligomer (SAPP). The anhydride bonds can be detected by Fourier transform infrared spectroscopy by the characteristic double peak at 1750 cm⁻¹ and 1820 cm⁻¹, with a corresponding disappearance of the carboxylic acid peak normally at 1700 cm⁻¹.

In one embodiment, the oligomers can be made from diacids described, for example, in U.S. Pat. Nos. 4,757,128, 4,997,904 and 5,175,235, the disclosures of which are incorporated herein by reference. For example, monomers such as sebacic acid, bis(p-carboxy-phenoxy)propane, isophthalic acid, fumaric acid, maleic acid, adipic acid or dodecanedioic acid can be used.

Organic dyes, because of their electronic charge and hydrophilicity or hydrophobicity, can alter the bioadhesive properties of a variety of polymers when incorporated into the polymer matrix or bound to the surface of the polymer. A partial listing of dyes that affect bioadhesive properties include, but are not limited to: acid fuchsin, alcian blue, alizarin red s, auramine o, azure a and b, Bismarck brown y, brilliant cresyl blue ald, brilliant green, carmine, cibacron blue 3GA, congo red, cresyl violet acetate, crystal violet, eosin b, eosin y, erythrosin b, fast green fcf, giemsa, hematoylin, indigo carmine, Janus green b, Jenner's stain, malachite green oxalate, methyl blue, methylene blue, methyl green, methyl violet 2b, neutral red, Nile blue a, orange II, orange G, orcein, paraosaniline chloride, phloxine b, pyronin b and y, reactive blue 4 and 72, reactive brown 10, reactive green 5 and 19, reactive red 120, reactive yellow 2,3, 13 and 86, rose bengal, safranin o, Sudan III and IV, Sudan black B and toluidine blue.

Polymers Functionalized with Hydroxy-Substituted Aromatic Groups

Polymers having an aromatic group which contains one or more hydroxyl groups grafted onto them or coupled to individual monomers are also suitable for use in the bioadhesive coatings of the invention, as described in U.S. Provisional Application No. 60/528,042, filed Dec. 9, 2003, U.S. application Ser. No. 11/009,327, filed Dec. 9, 2004, and WO 2005/056708, the contents of which are incorporated herein by reference. Such polymers can be biodegradable or non-biodegradable polymers. The polymer can be hydrophobic. Preferably, the aromatic group is catechol or a derivative thereof and the polymer contains reactive functional groups, so that a hydroxyl-substituted aromatic group can be readily attached. Typically, the polymer is a polyanhydride and the aromatic compound is the catechol derivative DOPA. These materials display bioadhesive properties superior to conventional bioadhesives used in therapeutic and diagnostic applications.

The molecular weight of the suitable polymers and percent substitution of the polymer with the aromatic group may vary greatly. The degree of substitution varies based on the desired adhesive strength, it may be as low as 10%, 25% or 50%, or up to 100% substitution. Generally, about 10 to about 40%, such as about 20% to about 30% of the monomers in the polymeric backbone are substituted with at least one aromatic group. The resulting polymer typically has a molecular weight ranging from about 1 to 2,000 kDa.

The polymer that forms that backbone of the bioadhesive material can be a biodegradable polymer. Examples of preferred biodegradable polymers include synthetic polymers such as poly hydroxy acids, such as polymers of lactic acid and glycolic acid, polyanhydrides, poly(ortho)esters, polyesters, polyurethanes, poly(butyric acid), poly(valeric acid), poly(caprolactone), poly(hydroxybutyrate), poly(lactide-co-glycolide) and poly(lactide-cocaprolactone), and natural polymers such as alginate and other polysaccharides, collagen and chemical derivatives thereof (substitutions, additions of chemical groups, for example, alkyl, alkylene, hydroxylations, oxidations, and other modifications routinely made by those skilled in the art), albumin and other hydrophilic proteins, zein and other prolamines and hydrophobic proteins, copolymers and mixtures thereof. In general, these materials degrade either by enzymatic hydrolysis or exposure to water in vivo and by surface or bulk erosion. The foregoing materials may be used alone, as physical mixtures (blends), or as co-polymers.

Suitable polymers can formed by first coupling the aromatic compound to the monomer and then polymerizing. In this example, the monomers may be polymerized to form a polymer backbone, including biodegradable and non-biodegradable polymers. Suitable polymer backbones include, but are not limited to, polyanhydrides, polyamides, polycarbonates, polyalkylenes, polyalkylene oxides such as polyethylene glycol, polyalkylene terephthalates such as poly(ethylene terephthalate), polyvinyl alcohols, polyvinyl ethers, polyvinyl esters, polyethylene, polypropylene, poly(vinyl acetate), poly(vinyl chloride), polystyrene, polyvinyl halides, polyvinylpyrrolidone, polyhydroxy acids, polysiloxanes, polyurethanes and copolymers thereof, alkyl cellulose, hydroxyalkyl celluloses, cellulose ethers, cellulose esters, nitrocellulloses, polymers of acrylic and methacrylic esters, methyl cellulose, ethyl cellulose, hydroxypropyl cellulose, hydroxy-propyl methyl cellulose, hydroxybutyl methyl cellulose, cellulose acetate, cellulose propionate, cellulose acetate butyrate, cellulose acetate phthalate, carboxyethyl cellulose, cellulose triacetate, cellulose sulfate sodium salt, and polyacrylates such as poly(methyl methacrylate), poly(ethylmethacrylate), poly(butylmethacrylate), poly(isobutylmethacrylate), poly(hexylmethacrylate), poly(isodecylmethacrylate), poly(lauryl methacrylate), poly(phenyl methacrylate), poly(methyl acrylate), poly(isopropyl acrylate), poly(isobutyl acrylate), poly(octadecyl acrylate).

A suitable polymer backbone can be a known bioadhesive polymer that is hydrophilic or hydrophobic. Hydrophilic polymers include CARBOPOL™, polycarbophil, cellulose esters, and dextran.

Non-biodegradable polymers, especially hydrophobic polymers, are also suitable as polymer backbones. Examples of preferred non-biodegradable polymers include ethylene vinyl acetate, poly(methacrylic acid), copolymers of maleic anhydride with other unsaturated polymerizable monomers, e.g., poly(butadiene maleic anhydride), polyamides, copolymers and mixtures thereof and dextran, cellulose and derivatives thereof.

Hydrophobic polymer backbones include polyanhydrides, poly(ortho)esters, and polyesters such as polycaprolactone. Preferably, the polymer is sufficiently hydrophobic that it is not readily water soluble. For example, the polymer may be soluble up to less than about 1% w/w in water, preferably about 0.1% w/w in water at room temperature or body temperature. In the most preferred embodiment, the polymer is a polyanhydride, such as a poly(butadiene maleic anhydride) or another copolymer of maleic anhydride. Polyanhydrides may be formed from dicarboxylic acids, as described in U.S. Pat. No. 4,757,128 to Domb et al., incorporated herein by reference. Suitable diacids include aliphatic dicarboxylic acids, aromatic dicarboxylic acids, aromatic-aliphatic dicarboxylic acid, combinations of aromatic, aliphatic and aromatic-aliphatic dicarboxylic acids, aromatic and aliphatic heterocyclic dicarboxylic acids, and aromatic and aliphatic heterocyclic dicarboxylic acids in combination with aliphatic dicarboxylic acids, aromatic-aliphatic dicarboxylic acids, and aromatic dicarboxylic acids of more than one phenyl group. Suitable monomers include sebacic acid (SA), fumaric acid (FA), bis(p-carboxyphenoxy propane (CPP), isophthalic acid (IPh), and dodecanedioic acid (DD).

A wide range of molecular weights are suitable for the polymer that forms the backbone of the bioadhesive material. The molecular weight may be as low as about 200 Da (for oligomers) up to about 2,000 kDa. Preferably the polymer has a molecular weight of at least 1,000 Da, more preferably at least 2,000 Da, most preferably the polymer has a molecular weight of up to 20 kDa or up to 200 kDa. The molecular weight of the polymer may be up to 2,000 kDa (e.g., 20 kDa to 1,000 kDa or 2,000 kDa).

The range of substitution on the polymer can vary greatly and depends on the polymer used and the desired bioadhesive strength. For example, a butadiene maleic anhydride copolymer that is 100% substituted with DOPA will have the same number of DOPA molecules per chain length as a 67% substituted ethylene maleic anhydride copolymer. Typically, the polymer has a percentage substitution ranging from 10% to 100%, more typically ranging from 20% to 30%.

The polymers and copolymers that form the backbone of the bioadhesive material typically include reactive functional groups that interact with the functional groups on the aromatic compound.

It is important that the polymer or monomer that forms the polymeric backbone contains accessible functional groups that easily react or interact with molecules contained in the aromatic compounds, such as amines and thiols. In a preferred embodiment, the polymer contains amino reactive moieties, such as aldehydes, ketones, carboxylic acid derivatives, cyclic anhydrides, alkyl halides, aryl azides, isocyanates, isothiocyanates, succinimidyl esters or a combination thereof.

Preferably, the aromatic compound containing one or more hydroxyl groups is catechol or a derivative thereof. Optionally the aromatic compound is a polyhydroxy aromatic compound, such as a trihydroxy aromatic compound (e.g., phloroglucinol) or a multihydroxy aromatic compound (e.g., tannin). The catechol derivative may contain a reactive group, such as an amino, thiol, or halide group. A preferred catechol derivative is 3,4-dihydroxyphenylalanine (DOPA), which contains a primary amine. Tyrosine, the immediate precursor of DOPA, which differs only by the absence of one hydroxyl group in the aromatic ring, can also be used. Tyrosine is capable of conversion (e.g., by hydroxylation) to the DOPA form. A particularly preferred aromatic compound is an amine-containing aromatic compound, such as an amine-containing catechol derivative (e.g., dopamine).

Two general methods are used to form the polymer product. In one example, a compound containing an aromatic group which contains one or more hydroxyl groups is grafted onto a polymer. In this example, the polymeric backbone is a biodegradable polymer. In a second example, the aromatic compound is coupled to individual monomers and then polymerized.

Any chemistry which allows for the conjugation of a polymer or monomer to an aromatic compound containing one or more hydroxyl groups can be used, for example, if the aromatic compound contains an amino group and the monomer or polymer contains an amino reactive group, this modification to the polymer or monomer is performed through a nucleophilic addition or a nucleophilic substitution reaction, such as a Michael-type addition reaction, between the amino group in the aromatic compound and the polymer or monomer. Additionally, other procedures can be used in the coupling reaction. For example, carbodiimide and mixed anhydride based procedures form stable amide bonds between carboxylic acids or phosphates and amino groups, bifunctional aldehydes react with primary amino groups, bifunctional active esters react with primary amino groups, and divinylsulfone facilitates reactions with amino, thiol, or hydroxy groups.

The aromatic compounds are grafted onto the polymer using standard techniques to form the bioadhesive material. In one example, L-DOPA is grafted to maleic anhydride copolymers by reacting the free amine in L-DOPA with the maleic anhydride bond in the copolymer.

A variety of different polymers can be used as the backbone of the bioadhesive material, as described above. Additional representative polymers include 1:1 random copolymers of maleic anhydride with ethylene, vinyl acetate, styrene, or butadiene. In addition, a number of other compounds containing aromatic rings with hydroxy substituents, such as tyrosine or derivatives of catechol, can be used in this reaction.

In another embodiment, the polymers are prepared by conjugate addition of a compound containing an aromatic group that is attached to an amine to one or more monomers containing an amino reactive group. In a preferred method, the monomer is an acrylate or the polymer is acrylate. For example, the monomer can be a diacrylate such as 1,4-butanediol diacrylate, 1,3-propanediol diacrylate, 1,2-ethanediol diacrylate, 1,6-hexanediol diacrylate, 2,5-hexanediol diacrylate or 1,3-propanediol diacrylate. In an example of the coupling reaction, the monomer and the compound containing an aromatic group are each dissolved in an organic solvent (e.g., THF, CH₂Cl₂, methanol, ethanol, CHCl₃, hexanes, toluene, benzene, CCl₄, glyme, diethyl ether, etc.) to form two solutions. The resulting solutions are combined, and the reaction mixture is heated to yield the desired polymer. The molecular weight of the synthesized polymer can be controlled by the reaction conditions (e.g., temperature, starting materials, concentration, solvent, etc) used in the synthesis.

For example, a monomer, such as 1,4-phenylene diacrylate or 1,4-butanediol diacrylate having a concentration of 1.6 M, and DOPA or another primary amine containing aromatic molecule are each dissolved in an aprotic solvent such as DMF or DMSO to form two solutions. The solutions are mixed to obtain a 1:1 molar ratio between the diacrylate and the amine group and heated to 56° C. to form a bioadhesive material.

### Coatings

Preferred bioadhesive coatings do not appreciably swell upon hydration, such that they do not substantially inhibit or block movement (e.g., of ingested food) through the gastrointestinal tract, as compared to the polymers disclosed by Duchene et al. Generally, polymers that do not appreciably swell upon hydration include one or more hydrophobic regions, such as a polymethylene region (e.g., (CH₂)ₙ, where n is 4 or greater). The swelling of a polymer can be assessed by measuring the change in volume when the polymer is exposed to an aqueous solution. Polymers that do not appreciably swell upon hydration expand in volume by 50% or less when fully hydrated. Preferably, such polymers expand in volume by less than 25%, less than 20%, less than 15%, less than 10% or less than 5%. Even more preferably, the bioadhesive coatings are mucophilic.

In one embodiment, a polymer that does not appreciably swell upon hydration (e.g., a hydrophobic polymer) is mixed or blended with a polymer that does swell or a hydrophilic substance (e.g., Carbopol™, poly(acrylic acid), small organic acids such as citric acid, maleic acid, fumaric acid, hydrophilic drugs, ionic and non-ionic detergents, sugars, salts such as NaCl, disintegrants), provided that the amount of swelling or hydration in the polymer does not substantially interfere with bioadhesiveness. Generally, the amount of swellable polymer or hydrophilic substance is selected to sufficiently hydrate the non-swellable polymer to enhance its bioadhesiveness. The weight ratio of swellable to non-swellable polymer or hydrophilic substance to non-swellable polymer can be varied in order to obtain a coating that combines a desired amount of swelling (e.g., for faster adhesion) with longer-lasting adhesion, such as from 5:1 to 1:5 or 2:1 to 1:2. For example, the swellable polymer and/or hydrophilic substance can comprise about 1% to about 30% by weight of a bioadhesive coating.

In one embodiment, the bioadhesive polymeric coating consists of two layers, an inner bioadhesive layer that does not substantially swell upon hydration and an outer bioadhesive layer that is readily hydratable and optionally bioerodible, such as one comprised of Carbopol™.

The bioadhesive polymers discussed above can be mixed with one or more plasticizers or thermoplastic polymers. Such agents typically increase the strength and/or reduce the brittleness of polymeric coatings. Examples of plasticizers include dibutyl sebacate, polyethylene glycol, triethyl citrate, dibutyl adipate, dibutyl fumarate, diethyl phthalate, ethylene oxide-propylene oxide block copolymers such as Pluronic™ F68 and di(sec-butyl) fumarate. Example of thermoplastic polymers include polyesters, poly(caprolactone), polylactide, poly(lactide-co-glycolide), methyl methacrylate (e.g., EUDRAGIT™), cellulose and derivatives thereof such as ethyl cellulose, cellulose acetate and hydroxypropyl methyl cellulose (HPMC) and large molecular weight polyanhydrides. The plasticizers and/or thermoplastic polymers are mixed with a bioadhesive polymer to achieve the desired properties. Typically, the proportion of plasticizers and thermoplastic polymers, when present, is from 0.5% to 40% by weight.

In one embodiment, the bioadhesive polymer coating, in a dry packaged form of a tablet, is a hardened shell.

A pharmaceutical dosage form (e.g., tablet or a drug-eluting device) can have one or more coatings in addition to the bioadhesive polymeric coating, e.g., covering the surface of the bioadhesive coating. These coatings and their thickness can, for example, be used to control where in the gastrointestinal tract the bioadhesive coating becomes exposed. In one example, the additional coating prevents the bioadhesive coating from contacting the mouth or esophagus. In another example, the additional coating remains intact until reaching the small intestine (e.g., an enteric coating).

Examples of coatings include methylmethacrylates, zein, cellulose acetate, cellulose phthalate, HMPC, sugars, enteric polymers, gelatin and shellac. Premature exposure of a bioadhesive layer or dissolution of a tablet in the mouth can be prevented with a layer or coating of hydrophilic polymers such as HPMC or gelatin.

Coatings used in tablets of the invention typically include a pore former to render the coating permeable to the drug.

Pharmaceutical dosage forms (e.g., tablets and drug-eluting devices) of the invention can be coated by a wide variety of methods. Suitable methods include compression coating, coating in a fluidized bed or a pan and hot melt (extrusion) coating. Such methods are well known to those skilled in the art.

### Multi-Layer Tablets

The invention also includes multi-layer tablets comprising a first, a second and a third layer, where each layer includes one or more drugs and one or more excipients, where the first layer forms the core of the table, the second layer is adjacent to one side of the first layer and the third layer is adjacent to the opposite side of the first layer. At least one layer of the tablet includes a hydrophobic excipient and at least one drug in the tablet is hygroscopic, deliquescent or both. Preferably, at least one hygroscopic and/or deliquescent drug and at least one hydrophobic excipient are present (e.g., blended together) in at least one layer of a tablet.

Exemplary hydrophobic excipients include celluloses, particularly cellulose acetate and ethyl cellulose, stearic acid, magnesium stearate, glycerol monostearate, fatty acids and salts thereof, monoglycerides, diglycerides, triglycerides, oil, colloidal silicon dioxide and talc.

Such tablets optionally include one excipient present in an amount sufficient to be at least partially rate-controlling with respect to release of the drug from the tablet. Typically, tablets that include a rate-controlling excipient (e.g., a rate-controlling polymer) contain about 30% to about 60% by weight of the rate-controlling excipient. Alternatively, the amount of rate-controlling excipient is selected relative to the amount of drug in the tablet. In such cases, the weight of the rate-controlling excipient is about two times to about five times, such as about two times to about three times greater than the weight of the drug.

Typically, the inner and outer layers contain different proportions of each component (including the drug(s)), thereby establishing a gradient-type composition. In an exemplary embodiment, the first (inner) layer contains the greatest weight percentage of the drug(s). Accordingly, the second and third layers and any additional layers present contain lesser amounts of drug. In multi-layer tablets having more than three layers (e.g., those having a fourth and optionally a fifth layer), the additional layers can, for example, contain no drug or contain successively lesser amounts of drug. In general, layers the same distance away from the first or inner layer will contain approximately equal amount of drug, such that the tablet is essentially symmetrical about the inner layer. For tablets containing two or more drugs, the drugs can both be present in one or more layers or the different drugs are present in separate layers (i.e., the drugs are not mixed together in one layer).

For drugs requiring absorption in buccal and sublingual regions of the GIT, bioadhesive tablets and particularly bioadhesive multiparticulates and nanoparticles are desirable. Drugs absorbed in these sites avoid first-pass metabolism by liver and degradation by GIT enzymes and harsh pH conditions typically present in the stomach and small intestine. Drugs absorbed in the buccal and sublingual compartments benefit from rapid onset of absorption, typically within minutes of dosing. Particularly suitable are bioadhesive particulates in fast-dissolving dosage forms, e.g., OraSolv (Cima Labs) that disintegrate within 30 sec after dosing and release the bioadhesive particles. Target release profiles include immediate release (IR) and combinations of zero-order controlled release (CR) kinetics and first-order CR kinetics. Preferably, pharmaceutical formulations targeting the buccal and sublingual regions are constructed such that the formulation disintegrates before passing into the esophagus.

For drugs requiring absorption in the stomach and upper small intestine and/or topical delivery to these sites, particularly drugs with narrow absorption windows, bioadhesive, gastroretentive drug delivery systems are the option of choice. Bioadhesive tablets and multiparticulates are formulated to reside for durations greater than 3 hrs and optimally greater than 4, 5 or even 6 hrs in the fed state. Drug release profiles from these systems are tailored to match the gastric residence times, so that greater than 85% of the encapsulated drug is released during the gastric residence time. Target release profiles include zero-order CR kinetics, first-order CR kinetics and combinations of IR and CR kinetics.

For drugs requiring absorption or topical delivery only in the small intestine, enteric-coated, bioadhesive drug delivery systems are preferred. Such systems are particularly well suited for topical delivery of therapeutics to Crohn's disease patients. Enteric-coated, bioadhesive tablets and multiparticulates are formulated to reside in the stomach for durations less than 3 hrs in the fed state and less than 1 hr in the fasted state, during which time less than 10% of the encapsulated drug is released, due to the enteric coating. Following gastric emptying, the enteric coating dissipates, revealing the underlying bioadhesive coating. Dissipation of the enteric coating is typically controlled by pH and/or time duration. Typical enteric polymers utilizing pH as a control are Eudragit polymers manufactured by Rohm America: Eudragit L100-55 dissolves at pH values greater than 5.5, typically found in duodenum; Eudragit L100 dissolves at pH values exceeding 6.0, typically found in jejunum; Eudragit S100 dissolves at pH values exceeding 7.0, typically found in ileum and the ileocecal junction.

Time may be used to control unmasking of the bioadhesive coating. Coatings that dissolve after 3 hrs when the dosage form is administered in the fed state and after 1-2 hrs when the dosage form is administered in the fasted state are suitable for bioadhesive delivery systems to small intestine. Erosion of soluble polymer layers is one means to achieve a time-triggered, enteric dissolution. Polymers such as HPMC, HPC, PVP, PVA or combinations of the above may be used as time-delayed, enteric coatings and timing of the dissolution of the coating can be increased by applying thicker coating weights.

Alternately, non-permeable coatings of insoluble polymers, e.g., cellulose acetate, ethylcellulose, can be used as enteric coatings for delayed/modified release (DR/MR) by inclusion of soluble pore formers in the coating, e.g., PEG, PVA, sugars, salts, detergents, triethyl citrate, triacetin, etc., at levels ranging from 0.5 to 50% w/w of the coating and most preferably from 5 to 25% w/w of the coating.

Also suitable are rupturable coating systems, e.g., Pulsincap, that use osmotic forces of swelling from hydrophilic polymers to rupture enteric membranes to reveal underlying bioadhesive coatings.

Target release profiles for the small intestine include: no more than 10% drug release during the first 3 hrs post-dosing followed by either IR kinetics, zero-order CR kinetics, first-order CR kinetics and combinations of IR and CR kinetics.

For drugs requiring absorption or topical delivery only in the lower small intestine and colon enteric-coated, bioadhesive drug delivery systems are preferred. Such systems are particularly well suited for topical delivery of therapeutics to patients with Inflammatory Bowel Disease (IBD) including Crohn's disease and Ulcerative Colitis. Enteric-coated, bioadhesive tablets and multiparticulates are formulated to reside in the stomach for durations less than 3 hrs in the fed state and less than 1 hr in the fasted state, during which time less than 10% of the encapsulated drug is released, due to the enteric coating. Following gastric emptying, the enteric coating dissipates, revealing the underlying bioadhesive coating. Suitable means of controlling dissipation include pH, time duration and enzymatic action of colonic bacteria. Typical of enteric polymers for delivery to the lower gastrointestinal tract utilizing pH as a control are Eudragit polymers manufactured by Rohm America: Eudragit S100 and FS dissolves at pH values exceeding 7.0, typically found in ileum and the ileocecal junction.

Time may be used to control unmasking the bioadhesive coating. Coatings that dissolve after 4-5 hrs when the dosage form is administered in the fasted state and after 5-8 hrs when the dosage form is administered in the fed state are suitable for bioadhesive delivery systems to the lower small intestine and colon. Erosion of soluble polymer layers is one means to achieve a time-triggered, enteric dissolution. Polymers such as HPMC, HPC, PVP, PVA or combinations of the above may be used as time-delayed, enteric coatings and timing of the dissolution of the coating can be increased by applying thicker coating weights.

Alternately, non-permeable coatings of insoluble polymers, e.g., cellulose acetate, ethylcellulose, can be used as enteric coatings for delayed/modified release (DR/MR) by inclusion of soluble pore formers in the coating, e.g., PEG, PVA, sugars, salts, detergents, triethyl citrate, triacetin, etc., at levels ranging from 0.5 to 50% w/w of the coating and most preferably from 5 to 25% w/w of the coating.

Also, coatings of polymers that are susceptible to enzymatic cleavage by colonic bacteria are another means of ensuring release to distal ileum and ascending colon. Materials such as calcium pectinate can be applied as coatings to tablets and multiparticulates and disintegrate in the lower gastrointestinal tract, due to bacterial action. Calcium pectinate capsules for encapsulation of bioadhesive multiparticulates are also available.

Target release profiles for the lower gastrointestinal tract include: no more than 10% drug release during the first 4-5 hrs (fasted state) and 5-8 hrs (fed state) hrs post-dosing followed by either IR kinetics, zero-order CR kinetics, first-order CR kinetics and combinations of IR and CR kinetics.

In certain aspects, multi-layer tablets of the invention exhibit an approximately zero-order release of drug in in vitro testing and/or in vivo administration. For formulations where delivery to the stomach is desired, zero-order release advantageously occurs over about 6-12 hours, particularly 8-10 hours. For formulations where delivery to the stomach and small intestine are desired, zero-order release advantageously occurs over about 8-16 hours, particularly 10-14 hours. For formulations where delivery to the small intestine and colon are desired, zero-order release advantageously occurs over about 16-30 hours, particularly 22-26 hours.

Multi-layer or gradient tablets can be assembled in several different ways. In one embodiment, the tablet comprises at least one solid inner layer and two solid outer layers, each comprising one or more drugs and one or more pharmaceutical polymers and/or pharmaceutical excipients. In order to produce a gradient effect, the amount of drug and/or excipient differs among the inner and outer layers. For example, the one or more inner layers can comprise at least 34% of the total amount of the drug in the tablet and one or more polymer(s) and/or excipients(s), and each of the two outer layers can comprise not more than 33% of the total amount of drug in the tablet and one or more polymer(s) and/or excipients(s). Such tablets can also be used to commence release of different drugs at different times, by inclusion of different drugs in separate layers.

In another embodiment, the multi-layer tablet consists of a solid inner layer and two solid outer layers, each comprising a drug and one or more pharmaceutical polymers or pharmaceutical excipients, wherein at least one polymer or excipient is hydrophobic. Tablets of this embodiment preferably provide approximately zero-order or linear release kinetics. In still another embodiment, the multi-layer tablet is enteric coated.

One or more layers of the tablet can contain permeation enhancers to provide permeability enhancement of drugs through mucosal lining of the gastrointestinal tract (GIT). An absorption enhancer facilitates the uptake of a drug across the gastrointestinal epithelium. Absorption enhancers include compounds that improve the ability of a drug to be solubilized in the aqueous environment in which it is originally released and/or in the lipophilic environment of the mucous layer lining of the intestinal walls. Absorption enhancers further include compounds that increase disorder of the hydrophobic region of the membrane exterior of intestinal cells, promote leaching of membrane proteins that results in increased transcellular transport, or widen the pore radius between cells for increased paracellular transport. Examples of absorption enliancers include sodium caprate, ethylenediamine tetra(acetic acid) (EDTA), citric acid, lauroylcarnitine, palmitoylcarntine, tartaric acid and other agents known to increase GI permeability. Other suitable absorption enhancers include sodium salicylate, sodium 5-methoxysalicylate, indomethacin, diclofenac, polyoxyethylene ethers, sodium laurylsulfate, quaternary ammonium compounds, sodium deoxycholate, sodium cholate, octanoic acid, decanoic acid, glyceryl-1-monooctanoate, glyceryl-1-monodecanoate, DL-phenylalanine ethylacetoacetate enamine, chlorpromazine, D-myristoyl-L-propyl-L-prolyl-glycinate, concanavaline A, DL-α-glycerophosphate, and 3-amino-1-hydroxypropylidene-1,1-diphosphonate.

Alternatively, or in addition, the tablet is coated to provide additional control over diffusion of the drug or exposure of the tablet to the gastrointestinal tract (e.g., with an enteric coating). The diffusion-limiting coating can be a pharmaceutically-accepted polymeric coating material, such as methylmethacrylates (Eudragits™, Rohm and Hass; Kollicoat™, BASF), zein, cellulose acetate, cellulose phthalate and hydroxypropylmethylcellullose. The coatings can be applied using a variety of techniques including fluidized-bed coating, pan-coating and dip-coating.

Multi-layer tablets of the invention can include a bioadhesive coating, as described above.

Separately or in combination with the bioadhesive coating, a bioadhesive (such as those described above) can be included in one or more layers of the tablet.

Multi-layer tablets of the invention are readily prepared. In one example, the drug(s) is/are mixed with a compressible sugar and granulated with a binder solution of compressible sugar in purified water. Subsequent to drying, the granules are mixed with different amounts of colloidal silicon dioxide (Cabosil™) and magnesium stearate. The granules are mixed in different proportions with stearic acid or monosterate (30, 50, 70%, for example) and then fed into a multilayer tableting machine (such as a Korsch or Fette tableting machine) to yield a trilayer tablet. Additional layers, often with varying amount of drug granules (e.g., greater drug concentration in the center layer and decreasing in each subsequent outer layer), can readily be added. In certain embodiments, the outermost layers do not include a drug.

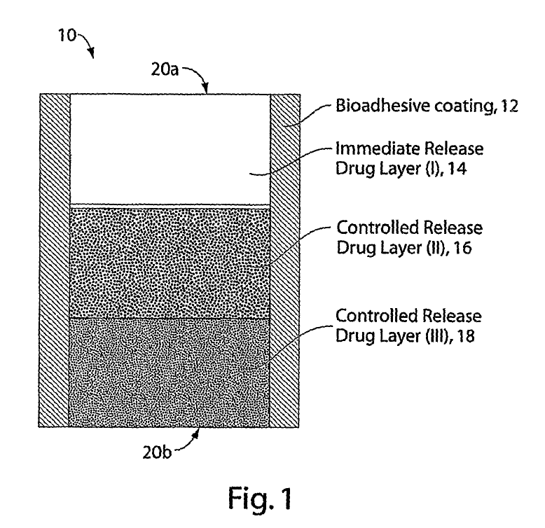

FIG. 1 illustrates a trilayer capsule shape tablet (10) including a first drug layer (14), second drug layer (16) and third drug layer (18). The capsule shape tablet (10) is partially enveloped in a bioadhesive polymeric plug (12) such that drug layer-ends (20a and 20b) remain exposed for drug release. A drug concentration gradient between the three drug layers allows a predetermined hybrid release profile to be achieved.

### General Characteristics

### Excipients

The cores of pharmaceutical dosage forms (e.g., tablets and drug-eluting devices) of the invention contain one or more excipients, carriers or diluents. These excipients, carriers or diluents can be selected, for example, to control the disintegration rate of a pharmaceutical dosage form (e.g., tablet or drug-eluting device). In particular, for bioadhesive tablets, it is advantageous for the time taken to release the contents of a tablet to be less than the gastrointestinal retention time or less than the retention time in the small and/or large intestines. In one embodiment, the release time of a tablet is at least 25% of the gastrointestinal, small intestine and/or large intestine retention time, at least 50% of the gastrointestinal, small intestine and/or large intestine retention time or at least 75% of the gastrointestinal, small intestine and/or large intestine retention time.

It will be understood by those skilled in the art that any vehicle or carrier conventionally employed and which is inert with respect to the active agent, and preferably does not interfere with bioadhesiveness in embodiments where that characteristic is desired, may be utilized for preparing and administering the pharmaceutical compositions of the present invention. Illustrative of such vehicles and carriers are those described, for example, in Remington's Pharmaceutical Sciences, 18th ed. (1990), the disclosure of which is incorporated herein by reference.

The formulations of the present invention for use in a subject comprise the drug, optionally together with one or more acceptable carriers or diluents therefor and optionally other therapeutic ingredients. The carriers or diluents must be “acceptable” in the sense of being compatible with the other ingredients of the formulation and not deleterious to the recipient thereof. The formulations can conveniently be presented in unit dosage form and can be prepared by any of the methods well known in the art of pharmacy. All methods include the step of bringing into association the drug with the carrier or diluent which constitutes one or more accessory ingredients. In general, the formulations are prepared by uniformly and intimately bringing into association the agent with the carriers and then, if necessary, dividing the product into unit dosages thereof.

Examples of carriers and diluents include pharmaceutically accepted hydrogels such as alginate, chitosan, methylmethacrylates, cellulose and derivatives thereof (microcrystalline cellulose, hydroxypropyl cellulose, hydroxypropyl methyl cellulose, carboxymethylcellulose, ethylcellulose), agarose and Povidone™, kaolin, magnesium stearate, starch, lactose, sucrose, density-controlling agents such as barium sulfate and oils, dissolution enhancers such as aspartic acid, citric acid, glutamic acid, tartartic acid, sodium bicarbonate, sodium carbonate, sodium phosphate, glycine, tricine and TRIS.

For multi-layer tablets in particular, the tablet typically includes at least one polymer or excipient. The polymer may be degradable or non-degradable. Suitable degradable polymers include polyesters, such as poly(lactic acid) (p[LA]), poly(lactide-co-glycolide) (p[LGA]), poly(caprolactone) (p[CL]); polyanhydrides such as poly(fumaric-co-sebacic anhydride) (p[FASA]) in molar ratios of 20:80 to 90:10, poly(carboxyphenoxypropane-co-sebacic anhydride) (p[CPPSA]), poly(adipic anhydride) (p[AA]); polyorthoesters; polyamides; and polyimides. Other suitable polymers include hydrogel-based polymers such as agarose, alginate, and chitosan. Suitable non-degradable polymers include polystyrene, polyvinylphenol, and polymethylmethacrylates (Eudragits™).

The excipients, carriers or diluents can also be selected to control the time until a pharmaceutical dosage form (e.g., tablet or drug-eluting device) detaches from a mucosal membrane. In particular, the addition of one or more disintegrating agents will reduce the time until a pharmaceutical dosage form (e.g., tablet or drug-eluting device) detaches. Alternatively or in combination with the disintegrating agents, an agent that interferes with the mucosa-tablet/device adhesion can be used to control the time until detachment occurs.

Suitable excipients include stabilizers, plasticizers, wetting agents, antitack agents, tack agents, foam agents, antifoam agents, binders, fillers, extenders, flavorants, dispersants, surfactants, solubilizers, solubilization inhibitors, glidants, lubricants, antiadherents, adherents, coatings, protective agents, sorbents, suspending agents, crystallization inhibitors, recrystallization inhibitors, disintegrants, acidulants, diluents, alkalizing agents, antioxidants, preservatives, colorants, electrolytes, solvents, antisolvents, accelerating agents, and/or retarding agents. Examples include alginate, chitosan, methylmethacrylates (Eudragits™), celluloses (especially microcrystalline cellulose, hydroxypropylmethylcellulose, ethylcellulose etc), agarose, Povidone™, lactose, microcrystalline cellulose, kaolin starch, magnesium stearate, stearic acid, glycerol monostearate, sucrose, compressible sugar, lactose and barium sulfate.

### Drugs and Active Agents

A wide variety of drugs can be included in pharmaceutical dosage forms (e.g., tablets and drug-eluting devices) of the invention. Such pharmaceutical dosage forms typically contain at least 1 mg of a drug. These pharmaceutical dosage forms can also contain at least 2 mg, at least 5 mg, at least 10 mg, at least 25 mg, at least 50 mg, at least 100 mg, at least 500 mg or at least 1000 mg of a drug (e.g., 2 mg to 1000 mg).

Drugs suitable for use herein can be small organic molecules (e.g., non-polymeric molecules having a molecular weight of 2000 Da or less, such as 1000 Da or less), peptides or polypeptides and nucleic acids.

Drugs may be classified using the Biopharmaceutical Classification System (BCS), which separates pharmaceuticals for oral administration into four classes depending on their solubility and their absorbability through the intestinal cell layer. According to the BCS, drug substances are classified as follows:

Class I—High Permeability, High Solubility

Class II—High Permeability, Low Solubility

Class III—Low Permeability, High Solubility

Class IV—Low Permeability, Low Solubility.

Drugs from these four classes can be used in the invention.

The interest in this classification system stems largely from its application in early drug development and then in the management of product change through its life-cycle. In the early stages of drug development, knowledge of the class of a particular drug is an important factor influencing the decision to continue or stop its development.

Class I drugs of the BCS system are highly soluble and highly permeable in the gastrointestinal (GI) tract. Sometimes BCS Class I drugs may be micronized to sizes less than 2 microns to increase the rate of dissolution.

Class II drugs are drugs that are particularly insoluble, or slow to dissolve, but that readily are absorbed from solution by the lining of the stomach and/or the intestine. Therefore, prolonged exposure to the lining of the GI tract is required to achieve absorption.

Many of the known Class II drugs are hydrophobic, and have historically been difficult to administer. Moreover, because of their hydrophobicity, there tends to be a significant variation in absorption depending on whether the patient is fed or fasted at the time of taking the drug. This in turn can affect the peak level of serum concentration, making calculation of dosage and dosing regimens more complex.

Class III drugs include biologic agents that have good water solubility and poor GI permeability, such as proteins, peptides, polysaccharides, nucleic acids, nucleic acid oligomers and viruses.

Class IV drugs are lipophilic drugs with poor GI permeability. Both Class III and IV drugs are often problematic or unsuitable for sustained release or controlled release. Class III and Class IV drugs are characterized by poor biomembrane permeability and are commonly delivered parenterally. Traditional approaches to parenteral delivery of poorly soluble drugs include using large volumes of aqueous diluents, solubilizing agents, detergents, non-aqueous solvents, or non-physiological pH solutions. These formulations, however, can increase the systemic toxicity of the drug composition or damage body tissues at the site of administration.

In one example, the drug is selected from hormones, enzymes, antigens, digestive aids, ulcer treatments (e.g., bismuth subsalicylate optionally in combination with antibiotics effective against H. pylori), antihypertensives, enzyme inhibitors, antiparasitics (e.g., antimalarials such as atovaquone), spermicides, anti-hemorrhoidal treatments, and radiopaque compounds. In another example, the drug is an antifungal agent (e.g., itraconazole, fluoconazole, terconazole, ketoconazole, saperconazole, griseofulvin, griseoverdin). In a further example, the drug is an antineoplastic agent. In yet another example, the drug is an antiviral agent (e.g., acyclovir). Other classes of drug suitable for inclusion in pharmaceutical dosage forms (e.g., tablets and drug-eluting devices) of the invention include steroids (e.g, danazol), immunosuppressants (e.g., cyclosporine), CNS active agents, cardiovascular agents, anti-depressant agents, anti-psychotic agents, anti-epileptic agents (e.g., carbamazepine), agents for treating a movement disorder (e.g., valproic acid and salts thereof) and anti-migraine agents (e.g., triptans such as sumatriptan).

The preferred materials to be incorporated into the bioadhesive pharmaceutical dosage forms (e.g., tablets or drug-eluting devices) are drugs and imaging agents. Drugs advantageously incorporate include antibiotics, antivirals (especially protease inhibitors alone or in combination with nucleosides for treatment of HIV or Hepatitis B or C), anti-parasites (helminths, protozoans), anti-cancer (referred to herein as “chemotherapeutics”, including cytotoxic drugs such as cisplatin and carboplatin, BCNU, 5FU, methotrexate, adriarnycin, camptothecin, and taxol), antibodies and bioactive fragments thereof (including humanized, single chain, and chimeric antibodies), antigen and vaccine formulations, peptide drugs, anti-inflammatories, and oligonucleotide drugs (including antisense, aptamers, ribozymes, external guide sequences for ribonuclease P, and triplex forming agents).

Examples of other useful drugs for use in bioadhesive pharmaceutical dosage forms (e.g., tablets and drug-eluting devices) include ulcer treatments such as Carafate™ from Marion Pharmaceuticals, neurotransmitters such as L-DOPA, antihypertensives or saluretics such as Metolazone from Searle Pharmaceuticals, carbonic anhydrase inhibitors such as Acetazolamide from Lederle Pharmaceuticals, insulin like drugs such as glyburide, a blood glucose lowering drug of the sulfonylurea class, synthetic hormones such as Android F from Brown Pharmaceuticals and Testred (methyltestosterone) from ICN Pharmaceuticals, and antiparasitics such as mebendzole (Vermox™, Jannsen Pharmaceutical).

Antigens can be microencapsulated in one or more types of bioadhesive polymer, and subsequently compressed into a tablet or filled into a capsule or the reservoir of a drug-eluting device, to provide a vaccine. The vaccines can be produced to have different retention times in the gastrointestinal tract. The different retention times, among other factors, can stimulate production of more than one type (IgG, IgM, IgA, IgE, etc.) of antibody.

In a preferred method for imaging, a radio-opaque material such as barium is coated with polymer. Radioactive materials or magnetic materials could be used in place of or in addition to the radio-opaque materials. Examples of other materials include gases or gas-emitting compounds that are radioopaque.

Bioadhesive pharmaceutical dosage forms (e.g., tablets and drug-eluting devices) of the invention are especially useful for treatment of inflammatory bowel diseases such as ulcerative colitis and Crohn's disease. In ulcerative colitis, inflammation is restricted to the colon, whereas in Crohn's disease, inflammatory lesions may be found throughout the gastrointestinal tract, from the mouth to the rectum. Sulfasalazine is one of the drugs that is used for treatment of the above diseases. Sulfasalazine is cleaved by bacteria within the colon to sulfapyridine, an antibiotic, and to 5-amino salicylic acid, an anti-inflammatory agent. The 5-amino salicylic acid is the active drug and it is needed locally. Direct administration of the degradation product (5-amino salicylic acid) may be more beneficial. A bioadhesive drug delivery system could improve the therapy by retaining the drug for a prolonged time in the intestinal tract. For Crohn's disease, retention of 5-aminosalicylic acid in the upper intestine is of great importance, since bacteria cleave the sulfasalazine in the colon, the only way to treat inflammations in the upper intestine is by local administration of 5-aminosalicylic acid.

Drugs particularly useful in the treatment of H. pylori include antibiotics such as amoxicillin, tetracycline, metronidazole and clarithromycin; H₂ blockers such as cimetidine, ranitidine, famotidine, and nizatidine; proton pump inhibitors such as omeprazole, lansoprazole, rabeprazole, esomeprazole, and pantoprozole; and stomach-lining protectors such as bismuth subsalicylate.

Multi-layer tablets of the invention are broadly useful for drug delivery, as they are compatible with a large number of different drugs. Suitable drugs include sodium valproate, valproic acid, divalproex sodium, antibiotics, non-steroidal anti-inflammatory drugs (“NSAIDS”), such as methyl salicylate, antiulcerative agents such as bismuth subsalicylate alone or in combination with antibiotics effective against organisms such as H. pylori, digestive supplements and cofactors, and vitamins.

In a preferred embodiment, the drug contains a valproic moiety. Sodium valproate is used for the treatment of generalized, partial or other epilepsy. Valproic acid is used for the treatment of generalized and partial seizures. Valproic acid and sodium valproate typically have a 1:1 dosing relationship. Side effects of treatment include occasional sedation (especially if given as part of polytherapy), ataxia and tremor and liver dysfunction; increased appetite with associated weight gain is the most common side effect. Nausea has been reported but is alleviated by taking the dose after food. The normal monotherapy dosage in adults is 600 mg daily in divided doses increased by 200 mg every 3 days until control is achieved up to a maximum of 2.5 g daily in divided doses. The usual dose range is 1-2 g daily in divided doses. The normal dose in children over 20 kg is 400 mg daily in divided doses, irrespective of weight, increased until control is achieved up to a maximum of 35 mg/kg/day in divided doses. The usual dose range 20-30 mg/kg/day in divided doses. The normal dose in children up to 20 kg is 20 mg/kg daily in divided doses. Preferably, multi-layer tablets of the invention (optionally coated with a bioadhesive) reduce or eliminate the need to administer multiple daily doses of valproate drugs. Generally, doses of valproate drugs are increased only if plasma concentrations are monitored. Because drugs such as phenyloin, carbamazepine and phenobarbitone increase the metabolism of sodium valproate, the dose required will be higher by 5-10 mg/kg/day. Once these agents have been withdrawn, the dose of sodium valproate can be reduced slightly as long as seizure control is maintained. Sustained release sodium valproate formulations are interchangeable with other dosage forms only when seizure control has been achieved, as long as the same total daily dose is given.

A class of drugs that is suitable for use in pharmaceutical dosage forms (e.g., tablets and drug-eluting devices), particularly the multi-layer tablets of the invention that include a hydrophobic excipient, are hygroscopic and/or deliquescent drugs. The term “hygroscopic” as used herein refers to substances that absorb significant amounts of atmospheric moisture when exposed to conditions of normal ambient relative humidity (RH), for example 10-50% RH. The term “deliquescent” refers to substances that tend to undergo gradual dissolution and/or liquefaction due to attraction/or absorption of moisture from air when exposed to these conditions. Those skilled the art will appreciate that over the usual range of ambient temperatures used in drug formulation, hygroscopicity and the state of deliquescence are largely temperature-independent, and that there are varying degrees of hygroscopicity and deliquescence.

Non-limiting examples of hygroscopic and/or deliquescent drugs suitable for use in the present invention include acetylcholine chloride, acetylcamitine, actinobolin, aluminum methionate, aminopentamide, aminopyrine hydrochloride, ammonium bromide, ammonium valerate, amobarbital sodium, anthiolimine, antimony sodium tartrate, antimony sodium thioglycollate, aprobarbital, arginine, aspirin, atropine N-oxide, avoparcin, azithromycin monohydrate, betahistine mesylate, betaine, bethanechol chloride, bismuth subnitrate, bupropion, butamirate, buthalital sodium, butoctamide, cacodylic acid, calcium chloride, calcium glycerophosphate, calcium iodide, carbachol, carnitine, caspofungin, ceruletide, chlorophyllin sodium-copper salt, choline alfoscerate, choline salicylate, choline theophyllinate, cilastatin, citicoline, cobalt dichloride, cromolyn disodium, cupric sulfate pentahydrate, cyanocobalamin, cyclobutyrol, cysteine hydrochloride, deaminooxytocin (L-isomer, anhydrous), deanol hemisuccinate, demecarium bromide, dexamethasone phosphate disodium salt, DL-dexpanthenol, dibucaine hydrochloride, dichlorophenarsine hydrochloride, diclofenac sodium, diethylcarbamazine citrate, dimethyl sulfoxidem, drotebanol, echinomycin, ephedrine (anhydrous), ergotamine, ethanolamine, fencamine hydrochloride, ferric chloride, ferrous iodide, ficin, gadobenate dimeglumine, gentamicin C complex sulfate, guanidine, heparin, hexadimethrine bromide, hexamethonium tartrate, hexobarbital sodium, histamine, hydrastine hydrochloride, hyoscyamine hydrobromide, S-[2-[(1-iminoethyl)amino]ethyl]-2-methyl-L-cysteine, imipramine N-oxide, isometheptene hydrochloride, isosorbide, levothyroxine sodium, lichenifonnins, lobeline sulfate, magnesium chloride hexahydrate, magnesium trisilicate, menadione, mercaptomerin sodium, mersalyl, metaraminol, methacholine chloride, methantheline bromide, methantheline chloride, methitural sodium, L-methyldopa sesquihydrate, methylmethioninesulfonium chloride, mildiomycin, minocycline hydrochloride, mitoxantrone dihydrochloride, morpholine, muscarine chloride, nafronyl acid oxalate, narceine, nicotine, nicotinyl alcohol, nolatrexed dihydrochloride, omeprazole, oryzacidin, oxalic acid, oxophenarsine hydrochloride, panthenol, pantothenic acid (sodium salt), papain, penicillamine hydrochloride, penicillin G (potassium salt), pentamethonium bromide, pentamidine isethionate, pepsin, perazine dihydrochloride, phenobarbital, sodium 5,5-diphenyl hydantoinate, phethenylate sodium, phosphocreatine (calcium salt tetrahydrate), physostigmine sulfate, pilocarpine hydrochloride, pipemidic acid, podophyllotoxin-beta-D-glucoside, potassium carbonate, potassium iodide, pralidoxime mesylate, prednisolone sodium phosphate, procainamide hydrochloride, procaine butyrate, L-proline, promazine hydrochloride, propamidine isethionate, prostacyclin sodium, pyridostigmine bromide, pyronaridine, quinacillin disodium, quinoline, radioactive sodium iodide, reserpilic acid dimethylaminoethyl ester dihydrochloride, secobarbital sodium, silver fluoride, sodium acetate, sodium bromide, sodium propionate, sodium dibunate, sodium dichromate(VI), sodium nitrite, sodium pentosan polysulfate, sodium valproate, soluble sulfamerazine, stibocaptate, streptomycin, succinylcholine bromide, succinylcholine iodide, sulfaquinoxaline, sulisatin disodium, suramin sodium, tamoxifen citrate, taurocholic acid, terazosin hydrochloride, thiobutabarbital sodium, thiopental sodium, ticarcillin disodium, 2,2,2-trichloroethanol, trientine, triethanolamine, triftazin, tolazoline hydrochloride, vinbarbital sodium, viomycin, vitamin B₁₂, zinc iodide, and combinations thereof, and pharmaceutically acceptable hygroscopic and/or deliquescent salts and variants thereof.

More than one type of drug can be present in a pharmaceutical dosage form (e.g., tablet or a drug-eluting device) of the invention. The drugs can be evenly distributed throughout a medicament or can be heterogeneously distributed in a medicament, such that one drug is fully or partially released before a second drug.

### Pharmaceutical Dosage Forms

Pharmaceutical dosage forms (e.g., tablets, capsules, drug-eluting devices) of the invention typically weigh at least 5 mg. Tablets, capsules and drug-eluting devices can also weigh at least 10 mg, at least 15 mg, at least 25 mg, at least 50 mg, at least 100 mg, at least 500 mg or at least 1000 mg. Typically, such objects weigh 10 mg to 500 mg.

The pharmaceutical dosage forms (e.g., capsules or tablets) typically contain between 10 and 70% of therapeutic, diagnostic or prophylactic agent (referred to generally as “drug”) by weight of a dosage form, or between 30 and 90% by weight of the core of a dosage form, where each coating makes up between 1-10%, preferably 5-6%, by weight of the dosage form, up to a total of about 30% by weight. The coating can include drug, in ratios of, for example, from 5 and 50% by weight of the coating, preferably between 20 and 40% by weight of the coating, while still retaining rate control.

Pharmaceutical dosage forms (e.g., tablets, capsules, drug-eluting devices) of the invention typically measure at least 2 mm in one direction. For example, pharmaceutical dosage forms can measure at least 5 mm, at least 10 mm, at least 15 mm or at least 20 mm in one direction. Typically, the diameter of the pharmaceutical dosage forms is 2 to 40 mm, preferably 10 to 30 mm such as 20 to 26 mm. Mini-tablets have a diameter of 2 mm to about 5 mm. Such pharmaceutical dosage forms can measure at least 2 mm, at least 5 mm, at least 10 mm, at least 15 mm or least 20 mm in a second direction and, optionally, a third direction. For example, pharmaceutical dosage forms of the invention ranges in size from about 2 to about 50 mm in length, from about 2 mm to about 15 mm in depth, and from about 2 mm to about 15 mm in width. Preferably, the pharmaceutical dosage form is of a size that facilitates swallowing by a subject.

The volume of a typical pharmaceutical dosage form of the invention is at least 0.008 mL, at least 0.01 mL, at least 0.05 mL, at least 0.1 mL, at least 0.125 mL, at least 0.2 mL, at least 0.3 mL, at least 0.4 mL or at least 0.5 mL, such as from 0.008 mL to 0.5 mL.

Suitable types of tablets are discussed in U.S. Provisional Applications Nos. 60/605,199, filed on Aug. 27, 2004, 60/605,198, filed on Aug. 27, 2004, and 60/635,812, filed on Dec. 13, 2004, the contents of which are incorporated by reference.

In one example, the tablet is a trilayer tablet having an inner core that includes one or more drugs in an appropriate matrix of excipients (e.g., HPMC, MCC, lactose) and is surrounded on two sides by a bioadhesive polymeric coating, which optionally is mixed with the one or more drugs. Preferred bioadhesive polymeric coatings are a DOPA-BMA (poly(butadiene-co-maleic acid)) polymer and a mixture of poly(fumaric-co-sebacic) anhydride and Eudragit™ RS PO.

In another example, the tablet is a longitudinally compressed tablet containing precompressed inserts of the drug and excipients and optionally a permeation enhancer. Drug is only released at the edge of this tablet, which can result in zero-order kinetics.

In yet another example, the tablet is comprised of a multiplicity of bioadhesive-coated microspheres that have been compressed into a tablet core and subsequently coated with a bioadhesive coating and one or more additional coatings (e.g., enteric coatings).

In a further example, the tablet includes a cavity through all or part of the tablet. A cavity extending through a tablet creates a channel open at both ends. Such tablets can be coated, for example, with a compression coating (e.g., enteric, bioadhesive, combinations, etc.) on all or selected surfaces. One example is a tablet having a channel through the tablet where the channel is uncoated.

In another example, the subject dosage formulations comprise an inner core, which comprises one or more drugs, excipients, and/or absorption enhancers that have been compressed to a form a solid, such as a tablet. For example, powdered drug formulations of the invention can be compressed to form a solid. In other embodiments, a drug can be used that in its pure form, under ambient conditions, is a liquid. In some embodiments, the liquid drug that is incorporated into a compressed inner core of the invention is present as a free base or free acid. In embodiments where the drug is a liquid drug (e.g., nicotine, valproic acid), the drug is preferably incorporated into a dosage form of the invention after it has been absorbed onto an absorbent material, such as kaolin clay or Cabosil (colloidal silicon dioxide).

In other embodiments, a solubilized form of an insoluble drug is incorporated into a dosage form of the invention. Solubilized forms of insoluble drugs may be aqueous-based or oil-based. For example, a water-insoluble drug may be dissolved in an organic solvent and then absorbed onto an absorbent material, such as a synthetic aluminosilicate or silicate, which can absorb certain organic solvents while still retaining the properties of a solid.

Capsules of the invention can be constructed in a multitude of ways. For examples, capsules can be filled with liquid, paste, powder, granules and/or beads. Granules and beads are optionally coated with a bioadhesive and/or other coating described herein. A capsule coated with a bioadhesive can either have the bioadhesive on the surface, or the bioadhesive can be coated with one or more layers that delay exposure of the bioadhesive to ambient conditions (e.g., to prevent the capsule from adhering to an upper portion or upper portion of the gastrointestinal tract). Capsules can include one or more excipients disclosed herein.

Capsules or tablets can be incorporated into standard pharmaceutical dosage forms such as gelatin capsules and tablets. Gelatin capsules, available in sizes 000, 00, 0, 1, 2, 3, 4, and 5, from manufacturers such as Capsugel®, may be filled with capsules or tablets and administered orally. Similarly, capsules or tablets may be dry blended or wet-granulated with diluents such as microcrystalline cellulose, lactose, colloidal silicon dioxide (e.g., Cabosil™) and binders such as hydroxypropylmethylcellulose, hydroxypropylcellulose, carboxymethylcellulose and directly compressed to form tablets.

Various drug-eluting devices are described in U.S. Pat. Nos. 4,290,426, 5,256,440, 5,378,475, 5,773,019 and 6,797,283, the contents of which are incorporated herein by reference.

In one example, the drug-eluting device includes an inner reservoir comprising the effective agent; a first coating layer, which is essentially impermeable to the passage of the effective agent; and a second coating layer, which is permeable to the passage of the effective agent. The first coating layer covers at least a portion of the inner reservoir; however, at least a small portion of the inner reservoir is not coated with the first coating layer (e.g., there are one or more pores in the first coating layer). The second coating layer essentially completely covers the first coating layer and the uncoated portion of the inner reservoir. Typically, the first coating layer is a non-bioerodable or a slowly bioerodable polymer (e.g., a polymer having a polymethylene backbone). For the present invention, the second coating can be either a bioadhesive polymeric coating or a coating between the first coating and the bioadhesive polymeric coating.

In another example, the drug-eluting device includes a multilayer core, often a bilayer, formed of polymer matrices that swell upon contact with the fluids of the stomach. At least one layer of the multilayer core includes a drug. A portion of the polymer matrices are surrounded by a band of insoluble material that prevents the covered portion of the polymer matrices from swelling and provides a segment of the dosage form that is of sufficient rigidity to withstand the contractions of the stomach. As a result, release of the drug is regulated by escape of the drug through one or more pores in the device. Typically, the core and the band of soluble material are coated with a bioadhesive polymeric coating.

In a further example, the drug-eluting device is an osmotic delivery system. Typically, the reservoir of such devices contains osmotic agents to draw water across a semi-permeable membrane and a swelling polymer to push drug out of the device at a controlled rate.

Preferably, drug-eluting devices of the invention release the drug contained therein with zero-order kinetics.

### EXEMPLIFICATION

### Example 1

### Fluoroscopy Study of Barium-Impregnated Trilayer Tablets with Bioadhesive Polymer Outer Layers

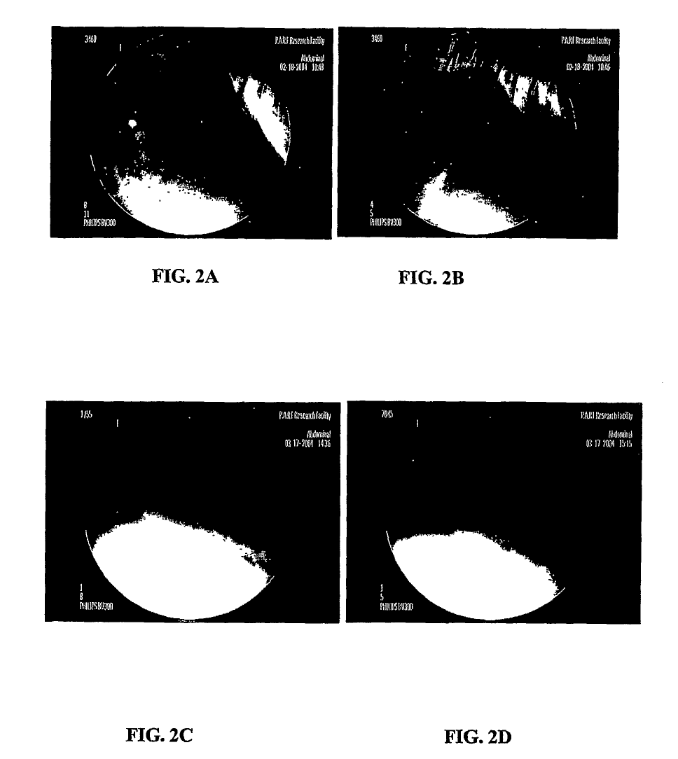

Trilayer tablets were prepared by sequentially filling a 0.3287×0.8937 “00 capsule” die (Natoli Engineering) with 333 mg of either Spheromer™ I or Spheromer™ III Bioadhesive polymer, followed by 233 mg of a blend of hydroxypropylmethylcellulose (HPMC) 4000 cps and 100 mg of barium sulfate, followed by an outer layer of 333 mg of either Spheromer™ I or III bioadhesive polymer. Trilayer tablets were prepared by direct compression at 2000 psi for 1 second using a Globepharma Manual Tablet Compaction Machine (MTCM-1). The tablets were administered to female beagles that were fasted for 24 hrs. The tablets were also dosed to fasted beagles that had been fed with chow, 30 min before dosing (fed). Tablets were continuously imaged with fluoroscopy over the course of 6 hrs in unrestrained dogs. Typical results are indicated below. Trilayer tablets with Spheromer™ I or III in the bioadhesive layers remained in the stomach of fasted dogs for up to 3.5 hrs and resided in the stomach of fed dogs in excess of 6 hrs, as shown in FIGS. 2A-D. The tablets did not mix with food contents and remained in contact with stomach mucosa at the same location until they passed into the small intestine.

### Example 2

### Fluoroscopy Study of Barium Impregnated Five-Layer Tablets with Spheromer™ I in Outer Layers

Five-layer tablets were prepared by sequentially filling a 0.3287×0.8937 “00 capsule” die (Natoli Engineering) with the following mixtures:

Composition of 5 Layer Tablet (1427 mg)

| Position | | mg/tablet | % w/w |
|---|---|---|---|
| Outer | Bioadhesive Layer (two) | 250 × 2 = 500 | |
| | | mg | |
| | poly[fumaric-co-sebacic]acid | | 66.2 |
| | 20:80 | | |
| | Eudragit RS PO | | 22.8 |
| | Sodium Chloride | | 10 |
| | Magnesium Stearate | | 1 |
| | | Total | 100% |
| Intermediate | Contrast Layer (two) | 100 × 2 = 200 | |
| | | mg | |
| | Barium sulfate | | 100% |
| Central | Core Layer (one) | 727 mg | |
| | 33% Itraconazole/Eudragit E100 | | 46 |
| | Microcrystalline Cellulose | | |
| | Granulation | | |
| | Spray Dried Lactose | | 13.7 |
| | HPMC, 5 cps | | 30 |
| | HPMC, 100 cps | | 10 |
| | Magnesium Stearate | | 0.3 |
| | | Total | 100% |

Five layer tablets, containing 100 mg of itraconazole, were prepared by direct compression at 3000 psi for 5 second using a Globepharma Manual Tablet Compaction Machine (MTCM-1). The tablets were administered to two female beagles that were fasted for 24 hrs and then fed chow 30 min before dosing (fed). Tablets were continuously imaged with fluoroscopy over the course of 8 hrs in unrestrained dogs. The tablets resided in the stomach of both fed dogs in excess of 6 hrs and ultimately split apart and passed into the small intestine at 8 hrs post-dosing. The tablets did not mix with food contents and remained in contact with stomach mucosa at approximately the same location until they passed into the small intestine. Pharmacokinetic results are indicated below:

| | Plasma | Itraconazole |
|---|---|---|
| Time | Mean | SD |
| (hr) | (ng/ml) | (ng/ml) |
| 0 | 0 | 0 |
| 1 | 98.2 | 62 |
| 4 | 984 | 277.2 |
| 8 | 1545 | 7.1 |
| 12 | 858 | 200.8 |
| 16 | 719 | 207.2 |
| 18 | 633.5 | 173.2 |
| 24 | 433.58 | 72.8 |
| 28 | 370 | 38.2 |
| 32 | 336 | 38.2 |
| 48 | 307.5 | 77.1 |
| AUC | 27409.3 | ng/ml*hr⁻¹ |
| Cₘₐₓ | 1545 | ng/ml |
| Tₘₐₓ | 8 | hrs |

By comparison, the innovator drug Sporanox® (Johnson & Johnson) is an immediate release dosage form containing 100 mg of itraconazole. When tested in the same beagle model, an AUC of 22,000 ng/ml*hr⁻¹, Cₘₐₓof 1200 ng/ml and Tₘₐₓ of 1.5 hrs were obtained. Itraconazole is a Biopharmaceutical Classification System Class 2 drug that has negligible water solubility and good GI permeability. It is slightly soluble in water at pH<3.5, limiting the site of GI absorption to duodenum and upper jejunum.

Clearly, the gastroretentive bioadhesive formulation described in this example delivered itraconazole for an extended period of time to the absorptive site in duodenum and upper jejunum. The 8 hr gastrointestinal residence time observed by fluoroscopy corresponds to the maximum itraconazole plasma concentration achieved at T max of 8 hrs.

### Example 3

### Sodium Valproate Tablets

Two different lots of sodium valproate bioadhesive tablet formulations, based on the concentration gradient approach, were prepared. Tablets from the first lot utilized L-Dopa/BMA (Spheromer™ III) as the bioadhesive polymer while tablets from the second lot were based on p(FA:SA) bioadhesive polymer. An additional tablet lot using ethyl cellulose as a non-bioadhesive polymer was also prepared. The following granulation and blending steps were used to make the three lots:

### Granulation

180.0 g of sodium valproate (Katwijk Chemie BV) were granulated using a binder solution prepared previously by dissolving 10 g of ethyl cellulose (10-FP, NF Premium) and 10 g of polyvinylpyrrolidone, K-15 in 667 mL of ethanol. Binder solution was applied onto the drug in a bench top fluidized-bed spray-coating unit (Vector Corp. model MFL.01). The following process parameters were used: fluid bed N₂ gas-flow=60-140 LPM; spray-nozzle pressure=15 psi; inlet temperature=50° C.; exhaust temperature=21-26° C.; pump speed=40 rpm; screen size=“I”; Wurster partition=medium; spray=bottom spray; and spray nozzle=medium. The granulation was dried and blended with 1% colloidal silicon dioxide. The granulation was stored in a 1-Liter glass jar containing DesiPak dessicant until used.

### Blending

The granulation sodium valproate was blended with various excipients to achieve the sodium valproate inner and outer layers compositions as shown below. The granulation was initially blended with ethyl cellulose or Spheromer™ I (p(FA:SA)) or Spheromer™ III (DOPA grafted on BMA) in a blender for 5 minutes followed by blending with magnesium stearate for additional 5 minutes.

### Composition of Common Inner Layer Blend

| Ingredients | Weight (%) |
|---|---|
| Sodium Valproate Granulation | 59.0 |
| Ethyl Cellulose, 10FP | 40.0 |
| Magnesium Stearate | 1.0 |
| Total | 100.0 |

### Composition of Outer Layer Blends

| Ingredients | Weight (%) |
|---|---|
| Sodium Valproate Granulation | 7.65 |
| Spheromer ™ I or Spheromer ™ | 91.35 |
| III | |
| Magnesium Stearate | 1.0 |
| Total | 100.0 |

### Trilayer Tablets

Trilayer tablets were compressed on the GlobePharma MCTM-1 manual tablet press using 0.328″×0.8937″ capsule shaped, deep concave punches. First 200 mg of outer blend was added to the die cavity and pre-compressed, then 987.2 mg of inner blend was added to the die cavity and pre-compressed again, and finally the 200 mg outer blend was added and compressed at 3000 psi for 1 s.

### In Vitro Dissolution Testing

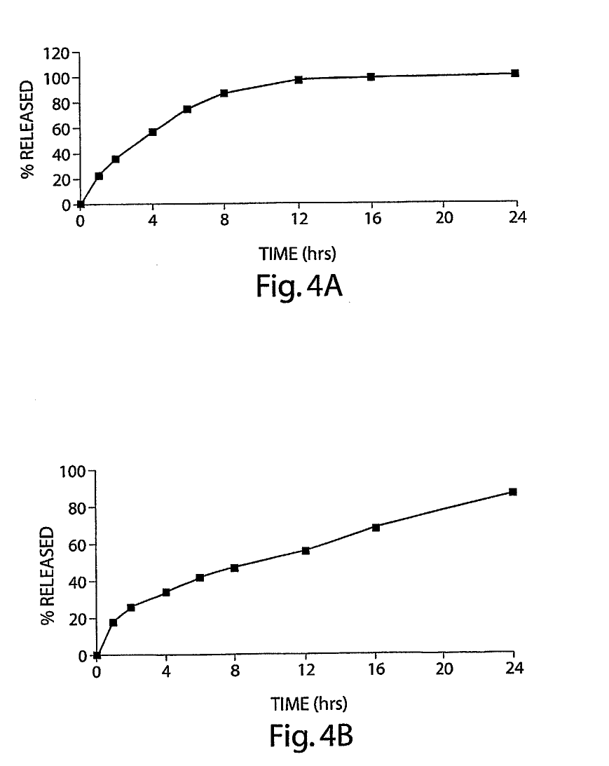

Trilayer tablets were tested for dissolution testing in a USP I apparatus using pH 6.8 PBS buffer at 100 rpm and 37° C. The dissolution profiles for the lots including Spheromer™ I and III are shown in FIGS. 4A and 4B, respectively. These dissolution profiles indicate that sodium valproate is released from the tablet in two phases, immediate release (outer layers) and sustained release (inner layer).

### Water Uptake Studies

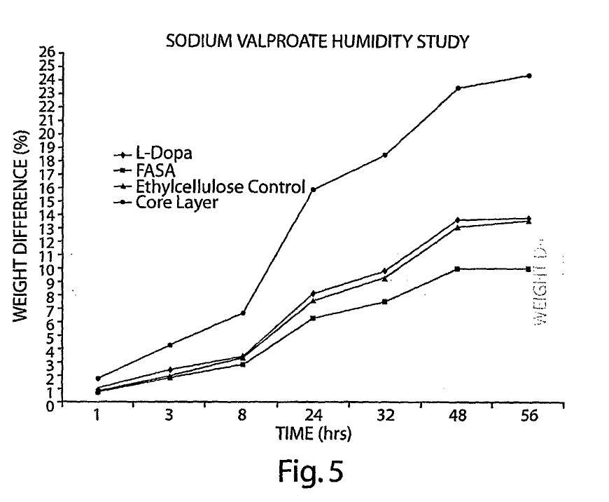

Sodium valproate trilayer tablet formulations (n=6) and a single-layer, matrix tablet formulation (n=6) consisting of only the core layer of the trilayer tablets were incubated at 45° C. and 60% relative humidity for up to 56 hours, where weight gain was measured at regular intervals. The results are illustrated in FIG. 5. The trilayer tablet formulations had considerably less water uptake compared to the unprotected core layer tablet. Uptake of water by tablets containing L-DOPA-BMA (Spheromer™ III) and ethylcellulose in the outer layers was approximately the same, whereas water uptake by tablets with p[FA:SA] polymer (Spheromer™ I) in the outer layers was approximately 30% less than the other trilayer tablet formulations. Thus, the use of hydrophobic polymers in multilayer tablet formulations provides important advantages for protection of hygroscopic drugs against water uptake.

### Example 4

### Carbidopa/Levodopa Tablets

Similar to the sodium valproate tablets described above, trilayer tablets containing carbidopa and levodopa were prepared. The contents of the inner and outer layers are as follows:

### Outer Layer Composition

| | Portion of Outer Layer | | Portion of Tablet |
|---|---|---|---|
| Ingredients | Weight (%) | Weight (mg) | Weight (mg) |
| Levodopa | 10.0 | 20.0 | 40.0 |
| Carbidopa, Monohydrate | 2.7 | 5.40 | 10.8* |
| Citric Acid | 2.0 | 4.0 | 8.0 |
| Spheromer ™ III | 84.3 | 168.6 | 337.2 |
| Magnesium Stearate | 1.0 | 2.0 | 4.0 |
| Total | 100.0 | 200.0 | 400.0 |

*Equivalent to 10 mg Carbidopa, anhydrous

### Inner Layer Composition

| Ingredients | Weight (%) | Weight (mg) |
|---|---|---|
| Levodopa | 40.0 | 160.0 |
| Carbidopa, Monohydrate | 10.79 | 43.16 |
| HPMC 100 cps | 38.0 | 152.0 |
| HPMC E5 | 4.71 | 18.84 |
| Glutamic Acid | 3.0 | 12.0 |
| Corn Starch | 3.0 | 12.0 |
| Magnesium Stearate | 0.5 | 2.0 |
| Total | 100.0 | 400.0 |

*Equivalent to 40 mg Carbidopa, anhydrous

For the inner and outer layer compositions, all ingredients, except magnesium stearate, were weighed and mixed thoroughly in a blender for five minutes. The mixed ingredients were blended with magnesium stearate for additional five minutes. 800 mg trilayer tablets were prepared as described in Example 3 using the GlobePharma Manual Tablet Compaction Machine (MTCM-1) equipped with standard 0.3287×0.8937″ punches. The tablets were compressed at compression force of 3000 psi for 1 s.

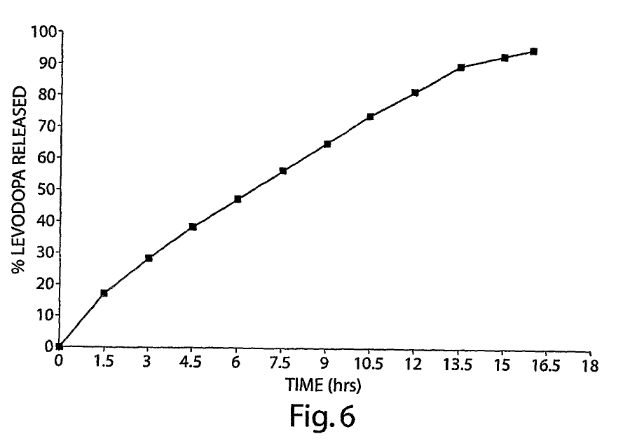

Bioadhesive tablets were tested for release profile in 0.1 N HCl at 37±0.5° C., in the USP II dissolution apparatus at 50 rpm. The in vitro release profile of levodopa from the trilayer tablets is shown in FIG. 6, which confirm that there is an immediate release of levodopa (from the outer layers), followed by a sustained release of levodopa (from the inner layer).

### Example 5

### Comparative Performance of Acyclovir Trilayer Tablet and Zovirax Tablets

Acyclovir is categorized as a Class 3 drug according to the Biopharmaceutical Classification system, because of its moderate water solubility and low bioavailability (10-20%). The drug is soluble only at acidic pH (pKa 2.27) limiting absorption in the gastrointestinal tract to duodenum and jejunum. There is no effect of food on drug absorption. Peak plasma levels are reached 3 to 4 hours following an oral dose. Bioavailability decreases with increasing drug dose. Elimination from plasma has a terminal half-life of 2.5 to 3.3 hours. Zovirax is normally dosed at either 200 mg every 4 hrs or 400 mg every 12 hrs, depending on the antiviral indication.

A trilayer tablet controlled release (CR) formulation includes an inner core and an outer bioadhesive coating. The controlled-release inner core blend contains 400 mg of acyclovir blended with glutamic acid, functioning as an acidulant, and Ethocel. The outer bioadhesive coating contains Spheromer™ III and excipients. The inner core blend is sandwiched between outer bioadhesive layers and direct compressed to create a bioadhesive, trilayer tablet.

The trilayer tablet is designed to reside in the stomach for greater than 6 hrs in the fed state and release acyclovir downstream, in a controlled manner, to the duodenum and upper jejunum, the main absorptive sites.

Cohorts of six, female beagle dogs (10-12 kg) were dosed with either Zovirax® or the trilayer tablet 30 min after a standard meal. 200 mg capsules of Zovirax® were dosed four times, every 6 hrs and compared to the trilayer tablets, containing 400 mg of acyclovir, dosed twice, every 12 hrs. The total drug dose in both cases was 800 mg administered over 24 hrs. 1 ml blood samples were collected at appropriate intervals extending to 48 hours. Plasma was collected after centrifugation for 10 min at 3,000 rpm and 4° C. Samples were stored frozen at −20° C. until analyzed.

Serum acyclovir was determined by LC/MS/MS. Turbulent flow chromatography using a 2300 HTLCTM system (Cohesive Technologies, Franklin, Mass.) was coupled to tandem-mass spectrometry (MS/MS) performed on a triple stage quadropole from Perkin Elmer SCIEX API 365 (Sciex, Concord, Ontario, Canada) with an atmospheric pressure ionization (API) chamber. The limit of detection of acyclovir in dog plasma was 10 ng/ml.

For each dog, the following pharmacokinetic parameters were calculated for the parent drug, acyclovir: maximum observed concentration (Cmax), time at which Cmax was observed (tmax), and area under the plasma concentration versus time curve (AUC) carried out to 48 hrs (AUC0-t).

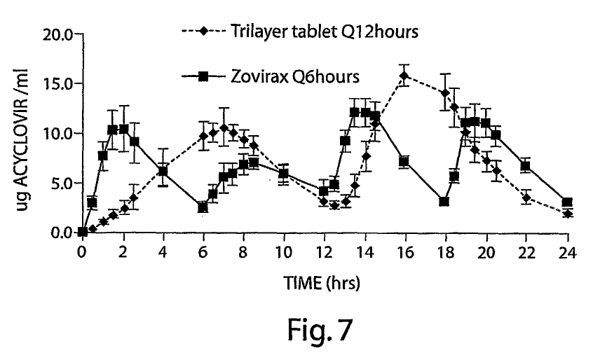

The effect of repeat dosing on plasma drug levels is shown in FIG. 7. The AUC's of the immediate release Zovirax capsules and the trilayer tablets were nearly identical (167 ug/ml*hr for Zovirax® compared to 164 ug/ml*hr for the trilayer tablets, n=6 dogs/study).

These data demonstrate that two doses of the trilayer tablet maintained plasma levels of acyclovir essentially as effectively as two doses of the Zovirax capsules. Thus, the trilayer tablets do not need to be administered as frequently as the Zorivax capsules.

### Example 6

### Pharmacokinetics of Bioadhesive Itraconazole Tablets Containing Bioadhesive Excipient

Bioadhesive, trilayer tablets containing 100 mg itraconazole in the central core layer were compressed using 0.3287×0.8937″ capsule-shaped dies (Natoli Engineering) at 3000 psi for 3 seconds in a GlobePharma Manual Tablet Compaction Machine (MTCM-1), as described above. The composition of the tablet was as follows:

Drug Layer Composition

| | | mg per | |
|---|---|---|---|
| Component | Function | tablet | % w/w |
| 30% Itraconazole/ | Drug/Polymer | 334 | 46.0 |
| Eudragit E100 Layered | Complex | | |
| onto Microcrystalline | | | |
| Cellulose (Emcocel 90 | | | |
| M) | | | |
| Hypromellose 100 cps | Rate-Controlling | 73 | 10.0 |
| | Polymer | | |
| Hypromellose 5 cps | Rate-Controlling | 218 | 30.0 |
| | Polymer | | |
| Spray-dried Lactose | Compressible binder | 100 | 13.7 |
| (Fast Flo 316) | | | |
| Magnesium Stearate | Lubricant | 2 | 0.3 |

| | | |
|---|---|---|
| Total | 727 | 100.0 |

Bioadhesive Layer Composition

| | | mg per | |
|---|---|---|---|
| Component | Function | tablet | % w/w |
| Poly (Fumaric-co- | Bioadhesive Polymer | 331 | 66.2 |
| sebacic)anhydride 20:80 | | | |
| polymer (Spheromer ™ I) | | | |
| Fumaric anhydride | Bioadhesive Excipient | 50 | 10.0 |
| oligomer (FAO) | | | |
| (Spheromer ™ II) | | | |
| Eudragit RSPO | Binder | 114 | 22.8 |
| Magnesium Stearate | Lubricant | 5 | 1.0 |
| Total | | 500 | 100.0 |

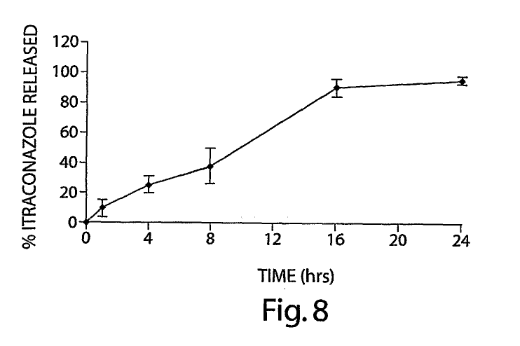

The trilayer tablets were tested for dissolution release profile in 900 mL of simulated gastric fluid (SGF), pH 1.2 in a USP II apparatus at 100 rpm. The results are shown in FIG. 8. Approximately 50% of the itraconazole was released within 8 hrs and 85% was released within 16 hrs. In contrast, dissolution of Sporanox® (approx. 85% drug release) occurred within 60 minutes.

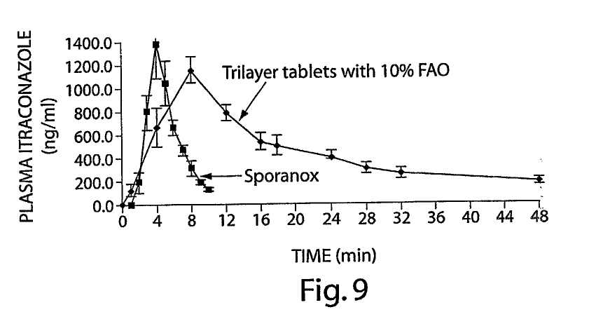

Sporanox® capsules and the bioadhesive trilayer tablets, each containing 100 mg of itraconazole, were administered to cohorts of six beagle dogs in the fed state and plasma levels of itraconazole were measured using LC/MS/MS. Pharmacokinetic profiles of two formulations in FIG. 9.

The area under the plasma itraconazole vs time curve (AUC), maximum concentration (Cmax) and time required to achieve Cmax (Tmax) were calculated and the results are indicated in the table below.

| | AUC | Cmax | Tmax |
|---|---|---|---|
| Formulation | (ng/mL*hr) | (ng/mL) | (hr) |
| Sporanox ® | 22741 | 1382.5 | 2.0 |
| Trilayer Tablets | 21292 | 1162.8 | 8.0 |

The bioadhesive trilayer tablets were able to achieve an AUC similar to that of the Sporanox® capsules. As per the Biopharmaceutical Classification System, itraconazole is a Class II drug, known to be absorbed only in the upper small intestine. The longer Tmax of the bioadhesive Spherazole formulation, compared to the Sporanox capsule, is characteristic of a controlled release formulation, as well as indicative of retention in the gastrointestinal tract.

### Example 7

### Bioadhesion of Coated Tablets

The bioadhesion of Spheromer™ II, Spheromer™ III, Gantrez® AN-119 BF (2,5-Furandione and methoxyethene) and hydrated Carbopol® 934P NF (cross-linked polyacrylic acid homopolymer) films, prepared by dip-coating on nylon supports, was tested using a Texture Analyzer TA XT II tensile tester, with pig intestine as the biological substrate. The parameters measured were fracture strength (peak force of detachment normalized for cross-sectional surface area) and tensile work (area under the deformation versus load curve).

Polymer films on supports were prepared by dip-coating in concentrated polymer solution and drying. Twenty percent (w/v) solutions were made for all test materials except for Carbopol® 934P, which was a 2% (w/v) solution in water. Spheromer™ II was dissolved with an equal amount of Eudragit® RL 100 in dichloromethane. The films on supports were air-dried for 24 hrs after dipping and lyophilized overnight to remove residual solvents.

Pig intestine was cut into at least 1 in² sections, mounted into a perforated, plastic holder with the mucus side up and submerged in phosphate buffered saline (PBS, pH 6.8). A fresh piece of tissue was used for each test. A polymer-coated support was mounted on the Texture Analyzer, and brought into contact with the pig intestine sample. An uncoated support was used as the control. After 7 minutes, the support was lifted away from the sample tissue and the load versus deformation curve was plotted. Instrumental settings are listed in the table below:

Texture Analyzer Settings

| CAPTION | VALUE | UNITS |
|---|---|---|
| Pre-Test Speed | 0.50 | mm/sec |
| Test Speed | 0.50 | mm/sec |
| Post-Test Speed | 0.50 | mm/sec |
| Force | 5.0 | g |
| Time | 420.00 | sec |
| Trigger Type | Button | — |
| Trigger Force | 5.0 | g |
| Trigger Distance | 0.000 | mm |

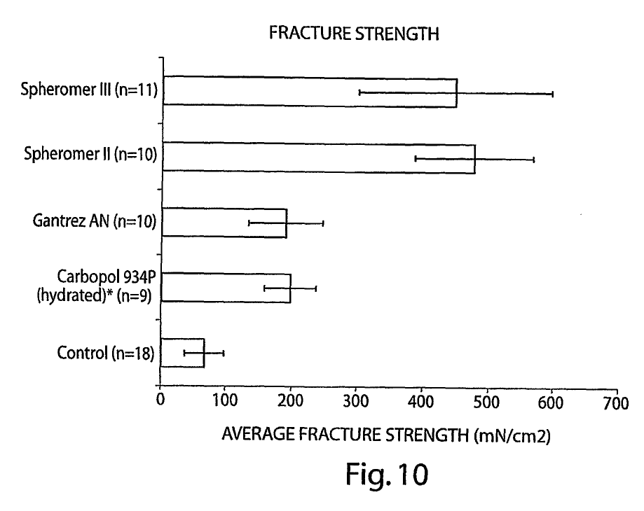

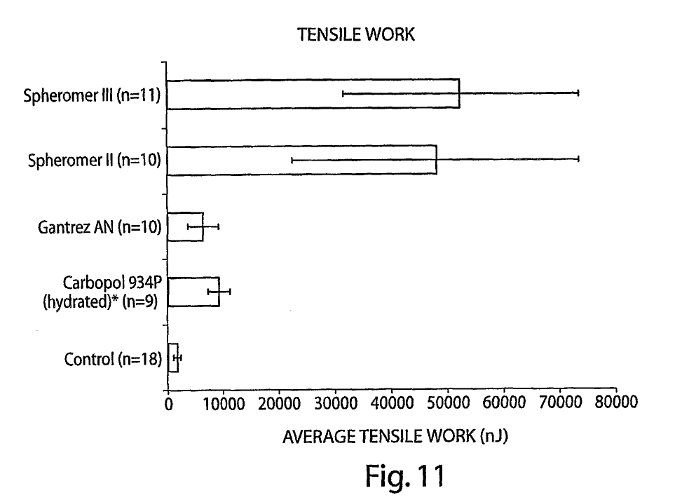

The results of the assay are shown in FIGS. 10 and 11.

While this invention has been particularly shown and described with references to preferred embodiments thereof, it will be understood by those skilled in the art that various changes in form and details may be made therein without departing from the scope of the invention encompassed by the appended claims.

1. A pharmaceutical dosage form for oral delivery of a drug, comprising a drug to be delivered gastrointestinally, and a bioadhesive polymeric coating, applied to at least a fraction of a surface of the dosage form, which coating provides the dosage form with a fracture strength of at least 100 N/m² as measured on rat intestine, and wherein the dosage form has a gastrointestinal retention time of at least 4 hours in a fed beagle dog model during which time said drug is released from said dosage form.

2. A tablet for oral delivery of a drug, comprising a core including a drug to be delivered gastrointestinally, and a bioadhesive polymeric coating, applied to at least one surface of the tablet, which coating provides the tablet with a fracture strength of at least 100 N/m² as measured on rat intestine, and wherein the tablet has a gastrointestinal retention time of at least 4 hours in a fed beagle dog model during which time said drug is released from said tablet.

3. The tablet of claim 2, wherein the bioadhesive polymeric coating does not substantially swell upon hydration.

4. The tablet of claim 2, wherein the tablet releases the drug in less than the gastrointestinal retention time.

5-9. (canceled)

10. The tablet of claim 2, wherein the bioadhesive polymeric coating is a synthetic polymer coating.

11. (canceled)

12. The tablet of claim 10, wherein the synthetic polymer coating is poly(fumaric-co-sebacic) anhydride.

13-14. (canceled)

15. The tablet of claim 2, wherein the bioadhesive polymeric coating comprises a polymer having a hydrophobic backbone and hydrophilic groups pendant from the backbone.

16. The tablet of claim 2, wherein the bioadhesive polymeric coating comprises a polymer having a hydrophobic backbone and hydrophobic groups pendant from the backbone.

17. A tablet for oral delivery of a drug, comprising a core including a drug to be delivered gastrointestinally, and a bioadhesive polymeric coating, applied to at least one surface of the tablet, which coating provides the tablet with a fracture strength of at least 100 N/m² as measured on rat intestine, wherein the tablet has a gastrointestinal retention time of at least 4 hours in a fed beagle dog model during which time said drug is released from said tablet, and wherein the bioadhesive polymer coating further includes metal compounds that enhance the mucosal adhesion of the polymer coating.

18. A tablet for oral delivery of a drug, comprising a core including a drug to be delivered gastrointestinally, and a bioadhesive polymeric coating, applied to at least one surface of the tablet, which coating provides the tablet with a fracture strength of at least 100 N/m² as measured on rat intestine, wherein the tablet has a gastrointestinal retention time of at least 4 hours in a fed beagle dog model during which time said drug is released from said tablet, and wherein the bioadhesive polymer coating further includes low molecular weight oligomers that enhance the mucosal adhesion of the polymer coating.

19. The tablet of claim 18, wherein the polymer coating further comprises metal compounds that enhance the mucosal adhesion of the polymer coating.

20. A tablet for oral delivery of a drug, comprising a core including a drug to be delivered gastrointestinally, and a bioadhesive polymeric coating, applied to at least one surface of the tablet, which coating provides the tablet with a fracture strength of at least 100 N/m² as measured on rat intestine, wherein the tablet has a gastrointestinal retention time of at least 4 hours in a fed beagle dog model during which time said drug is released from said tablet, and wherein the bioadhesive polymer coating comprises aromatic groups substituted with one or more hydroxyl groups.

21. A capsule for oral delivery of a drug, comprising a drug to be delivered gastrointestinally, and a bioadhesive polymeric coating, applied to at least one surface of the capsule, which coating provides the capsule with a fracture strength of at least 100 N/m² as measured on rat intestine, and wherein the capsule has a gastrointestinal retention time of at least 4 hours in a fed beagle dog model during which time said drug is released from said capsule.

22-33. (canceled)

34. The dosage form, tablet or capsule of any of claims 1, 2, 17 and 18, wherein the drug is an anti-migraine agent.

35. A drug-eluting device for oral delivery of a drug, comprising
a reservoir having a drug-containing core contained therein, and one or more orifices or exit ports through which drug from the core can elute from the device, and
a bioadhesive polymeric coating, applied to at least one surface of the device, which coating provides the device with a fracture strength of at least 100 N/m² as measured on rat intestine,
wherein the device has a gastrointestinal retention time of at least 4 hours in a fed beagle dog model during which time said drug is released from said device.

36. (canceled)

37. A tablet comprising a first, a second and a third layer, each layer comprising one or more drugs and one or more excipients, wherein the first layer forms the core of the table, the second layer is adjacent to one side of the first layer and the third layer is adjacent to the opposite side of the first layer, wherein at least one layer comprises a hydrophobic excipient and wherein at least one drug is hygroscopic, deliquescent or both.

38-54. (canceled)

55. A tablet comprising a first, a second and a third layer, wherein each layer comprises a drug and one or more excipients, wherein the first layer forms the core of the table, the second layer is adjacent to one side of the first layer and the third layer is adjacent to the opposite side of the first layer, and wherein the first layer comprises at least 34% of the total amount of the drug in the tablet and each of the second and third layers comprise not more than 33% each of the total amount of drug in the tablet.

56-63. (canceled)
